(19) United States
(12) Patent Application Publication
(10) Pub. No.: US 2010/0222412 A1
(43) Pub. Date: Sep. 2, 2010

(54) MODULATION OF GLUCOCORTICOID RECEPTOR EXPRESSION

(75) Inventors: Brett P. Monia, Encinitas, CA (US); Robert McKay, Poway, CA (US); Susan M. Freier, San Diego, CA (US); Sanjay Bhanot, Carlsbad, CA (US); Lynnetta Watts, Carlsbad, CA (US)

Correspondence Address: McDermott Will & Emery, 11682 EL CAMINO REAL, SUITE 400, SAN DIEGO, CA, 92130-2047, US

(73) Assignee: Isis Pharmaceuticals, Inc., Carlsbad, CA (US)

(21) Appl. No.: 12/687,533

(22) Filed: Jan. 14, 2010

Related U.S. Application Data
(63) Continuation of application No. 11/524,507, filed on Sep. 19, 2006, now abandoned.
(60) Provisional application No. 60/718,685, filed on Sep. 19, 2005.

(51) Int. Cl. A61K 31/7088; C07H 21/04; C12N 5/02; A61P 3/00; A61P 7/12; A61P 3/04

(52) U.S. Cl. 514/44 A; 536/24.5; 435/375

(57) ABSTRACT

Compounds, compositions and methods are provided for modulating the expression of glucocorticoid receptor. The compositions comprise antisense compounds, particularly antisense oligonucleotides which have particular in vivo properties, targeted to nucleic acids encoding glucocorticoid receptor. Methods of using these compounds for modulation of glucocorticoid receptor expression and for treatment of diseases are provided.

### CROSS-REFERENCE TO RELATED APPLICATIONS

This application is a Continuation Application of U.S. application Ser. No. 11/524,507, filed Sep. 19, 2006, which application claims priority under 35 USC 119(e) to U.S. Provisional Application Ser. No. 60/718,685 filed Sep. 19, 2005, all of which are herein incorporated by reference in their entirety.

### SEQUENCE LISTING

A computer-readable form of the sequence listing, on diskette, containing the file named BIOL0065USSEQ.txt, which is 37,122 bytes (measured in MS-DOS) and was created on Sep. 19, 2006, is herein incorporated by reference.

### FIELD OF THE INVENTION

Disclosed herein are compounds, compositions and methods for modulating the expression of glucocorticoid receptor in a cell, tissue or animal.

### BACKGROUND OF THE INVENTION

As increased gluconeogenesis is considered to be the major source of increased glucose production in diabetes, a number of therapeutic targets for the inhibition of hepatic glucose production have been investigated. Due to the ability of antagonists of the glucocorticoid receptor (also known as nuclear receptor subfamily 3, group C, member 1; NR3C1; GCCR; GCR; GRL; Glucocorticoid receptor, lymphocyte) to ameliorate diabetes in animal models, such compounds are among the potential therapies being explored. However, there are detrimental systemic effects of glucocorticoid receptor antagonists, including activation of the HPA axis (Link, Curr Opin Investig Drugs, 2003, 4, 421-429). Increased HPA axis activity is associated with suppression of immune-related inflammatory action, which can increase susceptibility to infectious agents and neoplasms. Conditions associated with suppression of immune-mediated inflammation through defects in the HPA axis, or its target tissues, include Cushing's syndrome, chronic stress, chronic alcoholism and melancholic depression (Chrousos, N Engl J Med, 1995, 332, 1351-1362). Thus, it is of great value to develop liver and fat-specific glucocorticoid receptor antagonists.

### SUMMARY OF THE INVENTION

The present invention is directed to oligomeric compounds targeted to and hybridizable with a nucleic acid molecule encoding GCCR which modulate the expression of GCCR. Provided herein are chimeric oligonucleotides referred to as “gapmers”, comprising a deoxynucleotide region or “gap” flanked on each of the 5′ and 3′ ends with “wings” comprised of one to four 2′-O-methoxyethyl nucleotides. The deoxynucleotide regions of the oligonucleotides of the invention are comprised of greater than ten deoxynucleotides, thus the gapmers of the present invention are “gap-widened” as compared to chimeric compounds comprising a ten deoxynucleotide gap region, such as are exemplified in US Publication US2005-0164271, which is herein incorporated by reference in its entirety. In some embodiments, as compared to oligonucleotides having the same sequence but comprising a ten deoxynucleotide region flanked on both the 5′ and 3′ ends with five 2′-O-(2-methoxyethyl) nucleotides, gap-widened oligonucleotides have comparable or improved potency without enhanced accumulation of oligonucleotide in the liver. Thus, embodiments of the present invention include gap-widened oligonucleotides targeting GCCR wherein potency is comparable to or better than that of an oligonucleotide having the same sequence but comprising a ten deoxynucleotide region flanked on both the 5′ and 3′ ends with five 2′-O-(2-methoxyethyl) nucleotides without enhanced accumulation of oligonucleotide in target tissues.

Another embodiment of the present invention includes gap-widened oligonucleotides targeting GCCR wherein kidney concentrations of said oligonucleotide are comparable to or decreased with respect to that of an oligonucleotide having the same sequence but comprising a ten deoxynucleotide region flanked on both the 5′ and 3′ ends with five 2′-O-(2-methoxyethyl) nucleotides while maintaining or improving potency in target tissues such as liver.

Further provided are methods of modulating the expression of GCCR in cells, tissues or, animals comprising contacting said cells, tissues or animals with one or more of the compounds or compositions of the present invention. For example, in one embodiment, the compounds or compositions of the present invention can be used to reduce the expression of GCCR in cells, tissues or animals.

In one embodiment, the present invention provides a method of treating a disease or condition mediated by glucocorticoid expression in an animal comprising contacting the animal with an effective amount of a compound of the invention. The diseases or conditions include diabetes, Type 2 diabetes, obesity, metabolic syndrome X, hyperglycemia, hyperlipidemia, or liver steatosis. In some embodiments, the hyperlipidemia is associated with elevated lipids such as blood cholesterol or elevated blood triglycerides. Blood lipids include plasma lipids and serum lipids. Further provided are methods of decreasing blood lipid levels, methods of reducing body fat mass, methods of decreasing liver triglyceride levels, and methods of improving insulin sensitivity in an animal by administering a compound of the invention.

Also provided is a method of decreasing blood glucose levels in an animal comprising administering a compound of the invention. The blood glucose levels may be fasting or fed glucose levels, and blood glucose levels include serum or plasma glucose levels. Further provided are methods of increasing insulin sensitivity and inhibiting hepatic glucose output.

Another aspect of the present invention is a method of delaying or preventing the onset of an increase in blood lipid or blood glucose levels in an animal by administering a compound of the invention.

The instant application is also related to U.S. Application No. 60/718,684, which is incorporated by reference in its entirety. The instant application is also related to U.S. application Ser. No. 11/231,243 and PCT Application No. PCT/US2005/033837, each of which is herein incorporated by reference in its entirety.

### DETAILED DESCRIPTION

### Overview

Disclosed herein are oligomeric compounds, including antisense oligonucleotides and other antisense compounds for use in modulating the expression of nucleic acid molecules encoding GCCR. This is accomplished by providing oligomeric compounds which hybridize with one or more target nucleic acid molecules encoding GCCR.

In accordance with the present invention are compositions and methods for modulating the expression of GCCR (also known as glucocorticoid receptor; nuclear receptor subfamily 3, group C, member 1; GR; GRL; and NR3C1). Listed in Table 1 are GENBANK® accession numbers of sequences which may be used to design oligomeric compounds targeted to GCCR. Oligomeric compounds of the invention include oligomeric compounds which hybridize with one or more target nucleic acid molecules shown in Table 1, as well as oligomeric compounds which hybridize to other nucleic acid molecules encoding GCCR.

The oligomeric compounds may target any region, segment, or site of nucleic acid molecules which encode GCCR. Suitable target regions, segments, and sites include, but are not limited to, the 5′UTR, the start codon, the stop codon, the coding region, the 3′UTR, the 5′ cap region, introns, exons, intron-exon junctions, exon-intron junctions, and exon-exon junctions.

TABLE 1
Gene Targets

| | GENBANK ® Accession Number | |
|---|---|---|
| Species | or Description | SEQ ID NO |
| Human | NM_000176.1 | 1 |
| Mouse | NM_012576.1 | 2 |
| Rat | NM_008173.1 | 3 |

The locations on the target nucleic acid to which active oligomeric compounds hybridize are herein below referred to as “validated target segments.” As used herein the term “validated target segment” is defined as at least an 8-nucleobase portion of a target region to which an active oligomeric compound is targeted. While not wishing to be bound by theory, it is presently believed that these target segments represent portions of the target nucleic acid which are accessible for hybridization.

The present invention includes oligomeric compounds which are chimeric compounds. An example of a chimeric compound is a gapmer having a 2′-deoxynucleotide region or “gap” flanked by non-deoxynucleotide regions or “wings”. While not wishing to be bound by theory, the gap of the gapmer presents a substrate recognizable by RNase H when bound to the RNA target whereas the wings are not an optimal substrate but can confer other properties such as contributing to duplex stability or advantageous pharmacokinetic effects. Each wing can be one or more non-deoxy oligonucleotide monomers. In one embodiment, the gapmer is comprised of a sixteen 2′-deoxynucleotide region flanked on each of the 5′ and 3′ ends by wings of two 2′-β-(2-methoxyethyl) nucleotides. This is referred to as a 2-16-2 gapmer. Thus, the “motif” of this chimeric oligomeric compound or gapmer is 2-16-2. In another embodiment, all of the internucleoside linkages are phosphorothioate linkages. In another embodiment the cytosines of the gapmer are 5-methylcytosines.

Embodiments of the present invention include oligomeric compounds comprising sequences of 13 to 26 nucleotides in length comprising a deoxy nucleotide region greater than 10 nucleobases in length flanked on each of its 5′ and 3′ ends with at least one 2′-O-(2-methoxyethyl) nucleotide. The preferred “gap-widened” oligonucleotides comprise 11, 12, 13, 14, 15, 16, 17, or 18 deoxynucleotides in the gap portion of the oligonucleotide. Preferred 5′ and 3′ flanking regions comprise 1, 2, 3, or 4 2′-O-(2-methoxyethyl) nucleotides. Preferred gap-widened gapmers have motifs including 1-18-1, 1-17-2, 2-17-1, 2-16-2, 3-14-3, and 4-12-4.

In preferred embodiments the oligomeric compounds target or hybridize with GCCR RNA. In another embodiment, the oligomeric compounds reduce the expression of GCCR RNA. In other embodiments, the oligomeric compounds reduce the expression of GCCR wherein the expression of GCCR is reduced by at least 10%, by at least 20%, by at least 30%, by at least 35%, by at least 40%, by at least 45%, by at least 50%, by at least 55%, by at least 60%, by at least 65%, by at least 70%, by at least 75%, by at least 80%, by at least 85%, by at least 90%, by at least 95%, or by 100%.

Oligonucleotides of the present invention include those wherein kidney concentrations of said oligonucleotide are decreased with respect to an oligonucleotide having the same sequence but comprising a ten deoxynucleotide region flanked on both the 5′ and 3′ ends with five 2′-β-(2-methoxyethyl) nucleotides. Oligonucleotides of the present invention include those wherein kidney concentrations of said oligonucleotide are comparable to or decreased with respect to those of an oligonucleotide having the same sequence but comprising a ten deoxynucleotide region flanked on both the 5′ and 3′ ends with five 2′-O-(2-methoxyethyl) nucleotides. Oligonucleotides of the present invention include those wherein potency with regard to target reduction or a therapeutic effect is comparable to or better than that of an oligonucleotide having the same sequence but comprising a ten deoxynucleotide region flanked on both the 5′ and 3′ ends with five 2′-O-(2-methoxyethyl) nucleotides without enhanced accumulation of oligonucleotide in target tissues. Preferred target tissues include liver, and adipose tissue.

The present invention provides antisense oligonucleotides 13 to 26 nucleobases in length targeted to a nucleic acid molecule encoding GCCR wherein the oligonucleotide comprises a first region, a second region, and a third region, wherein said first region comprises at least 11 deoxynucleotides and wherein said second and third regions comprise 1 to 4 2′-O-(2-methoxyethyl) nucleotides, said second and third regions flanking the first region on the 5′ and 3′ ends of said first region.

In some embodiments, oligonucleotides of the invention specifically hybridize to GCCR and reduce expression of GCCR. In some embodiments, the “gap” region comprises 11, 12, 13, 14, 15, 16, 17, or 18 nucleobases. In some embodiments, the antisense oligonucleotides are 20 nucleobases in length.

The oligomeric compounds can comprise about 8 to about 80 nucleobases (i.e. from about 8 to about 80 linked nucleosides), preferably between about 13 to about 26 nucleobases. One of ordinary skill in the art will appreciate that the preferred oligomeric compounds contemplated include compounds that are 13, 14, 15, 16, 17, 18, 19, 20, 21, 22, 23, 24, 25, or 26 nucleobases in length.

Compounds of the invention include oligonucleotide sequences that comprise at least the 8 consecutive nucleobases from the 5′-terminus of one of the illustrative antisense compounds (the remaining nucleobases being a consecutive stretch of the same oligonucleotide beginning immediately upstream of the 5′-terminus of the antisense compound which is specifically hybridizable to the target nucleic acid and continuing until the oligonucleotide comprises about 13 to about 26 nucleobases). Other compounds are represented by oligonucleotide sequences that comprise at least the 8 consecutive nucleobases from the 3′-terminus of one of the illustrative antisense compounds (the remaining nucleobases being a consecutive stretch of the same oligonucleotide beginning immediately downstream of the 3′-terminus of the antisense compound which is specifically hybridizable to the target nucleic acid and continuing until the oligonucleotide comprises about 13 to about 26 nucleobases). It is also understood that compounds may be represented by oligonucleotide sequences that comprise at least 8 consecutive nucleobases from an internal portion of the sequence of an illustrative compound, and may extend in either or both directions until the oligonucleotide contains about 13 to about 26 nucleobases.

Oligonucleotides of the invention include antisense oligonucleotides 20 nucleobases in length targeted to a nucleic acid molecule encoding GCCR and comprising at least an 8-nucleobase portion of SEQ ID NO: 34, 33, 35, 36, 37, 42, 45, 56, 61, 63, or 96. In one embodiment, oligonucleotides of the invention are antisense oligonucleotides 20 nucleobases in length targeted to a nucleic acid molecule encoding GCCR and having the sequence of SEQ ID NO: 34, 33, 35, 36, 37, 42, 45, 56, 61, 63, or 96. In one embodiment, oligonucleotides of the invention have the nucleobase sequence of SEQ ID NO: 37.

The present invention provides antisense oligonucleotides comprising the nucleobase sequence of SEQ ID NO: 37. In one embodiment, the oligonucleotides of the invention comprise at least an 8-nucleobase portion of the nucleobase sequence of SEQ ID NO: 37.

In one embodiment, the present invention provides antisense oligonucleotides 20 nucleobases in length targeted to a nucleic acid molecule encoding GCCR and comprising at least an 8-nucleobase portion of SEQ ID NO: 34, 33, 35, 36, 37, 42, 45, 56, 61, 63, or 96 wherein the oligonucleotide comprises a deoxynucleotide region 12, 13, 14, 15, 16, 17, or 18 nucleobases in length which is flanked on its 5′ and 3′ ends with 1 to 4 2′-O-(2-methoxyethyl) nucleotides and wherein the oligonucleotide specifically hybridizes to and reduces expression of GCCR RNA.

In one embodiment, the flanking regions are symmetrical (having the same number of nucleotides in the 5′ flanking region as in the 3′ flanking region). In another embodiment, the flanking regions are non-symmetrical (having a different number of nucleotides in the 5′ flanking region compared to the 3′ flanking region).

Antisense oligonucleotides of the invention may contain at least one modified internucleoside linkage. Modified internucleoside linkages include phosphorothioate linkages. The antisense oligonucleotides of the invention may also contain at least one modified nucleobase. In preferred embodiments, at least one cytosine is a 5-methylcytosine.

In other embodiments, the present invention includes antisense oligonucleotides having the nucleobase sequence of SEQ ID NO: 37, wherein the antisense oligonucleotide is characterized by a 12-deoxynucleotide region flanked on its 5′ and 3′ ends with four 2′-O-(2-methoxyethyl) nucleotides, a 14-deoxynucleotide region flanked on its 5′ and 3′ ends with three 2′-O-(2-methoxyethyl) nucleotides, a 16-deoxynucleotide region flanked on its 5′ and 3′ ends with two 2′-O-(2-methoxyethyl) nucleotides, a 17-deoxynucleotide region flanked on its 5′ and 3′ ends with one or two 2′-O-(2-methoxyethyl) nucleotides, or an 18-deoxynucleotide region flanked on its 5′ and 3′ ends with one 2′-O-(2-methoxyethyl) nucleotides.

In a particular embodiment, the antisense oligonucleotides have the nucleobase sequence of SEQ ID: 37, wherein the antisense oligonucleotide has a 12-deoxynucleotide region flanked on its 5′ and 3′ ends with four 2′-O(2-methoxyethyl) nucleotides. In a further embodiment, the antisense oligonucleotide specifically hybridizes to and reduces expression of GCCR. In a further embodiment, at least one internucleoside linkage is a phosphorothioate linkage. In a further embodiment, at least one cytosine is a 5-methylcytosine.

In a particular embodiment, the antisense oligonucleotide has the nucleobase sequence of SEQ ID: 37, wherein the antisense oligonucleotide has a 14-deoxynucleotide region flanked on its 5′ and 3′ ends with three 2′-O(2-methoxyethyl) nucleotides. In a further embodiment, the antisense oligonucleotide specifically hybridizes to and reduces expression of GCCR. In a further embodiment, at least one internucleoside linkage is a phosphorothioate linkage. In a further embodiment, at least one cytosine is a 5-methylcytosine.

In a particular embodiment, the antisense oligonucleotide has the nucleobase sequence of SEQ ID: 37, wherein the antisense oligonucleotide has a 16-deoxynucleotide region flanked on its 5′ and 3′ ends with two 2′-O(2-methoxyethyl) nucleotides. In a further embodiment, the antisense oligonucleotide specifically hybridizes to and reduces expression of GCCR. In a further embodiment, at least one internucleoside linkage is a phosphorothioate linkage. In a further embodiment, at least one cytosine is a 5-methylcytosine.

In a particular embodiment, the antisense oligonucleotide has the nucleobase sequence of SEQ ID: 37, wherein the antisense oligonucleotide has a 17-deoxynucleotide region flanked on its 5′ and 3′ ends with one or two 2′-O(2-methoxyethyl) nucleotides. In a further embodiment, the antisense oligonucleotide specifically hybridizes to and reduces expression of GCCR. In a further embodiment, at least one internucleoside linkage is a phosphorothioate linkage. In a further embodiment, at least one cytosine is a 5-methylcytosine.

In a particular embodiment, the antisense oligonucleotide has the nucleobase sequence of SEQ ID: 37, wherein the antisense oligonucleotide has a 18-deoxynucleotide region flanked on its 5′ and 3′ ends with one 2′-O(2-methoxyethyl) nucleotides. In a further embodiment, the antisense oligonucleotide specifically hybridizes to and reduces expression of GCCR. In a further embodiment, at least one internucleoside linkage is a phosphorothioate linkage. In a further embodiment, at least one cytosine is a 5-methylcytosine.

In another embodiment the antisense oligonucleotides comprise the nucleobase sequence of SEQ ID NO: 33. In one embodiment, the oligonucleotides of the invention comprise at least an 8-nucleobase portion of the nucleobase sequence of SEQ ID NO: 33.

In other embodiments, the present invention includes antisense oligonucleotides having the nucleobase sequence of SEQ ID NO: 33, wherein the antisense oligonucleotide is characterized by a 12-deoxynucleotide region flanked on its 5′ and 3′ ends with four 2′-O-(2-methoxyethyl) nucleotides, a 14-deoxynucleotide region flanked on its 5′ and 3′ ends with three 2′-O-(2-methoxyethyl) nucleotides, a 16-deoxynucleotide region flanked on its 5′ and 3′ ends with two 2′-O-(2-methoxyethyl) nucleotides, a 17-deoxynucleotide region flanked on its 5′ and 3′ ends with one or two 2′-O-(2-methoxyethyl) nucleotides, or an 18-deoxynucleotide region flanked on its 5′ and 3′ ends with one 2′-O-(2-methoxyethyl) nucleotides.

In a particular embodiment, the antisense oligonucleotides have the nucleobase sequence of SEQ ID: 33, wherein the antisense oligonucleotides have a 12-deoxynucleotide region flanked on its 5′ and 3′ ends with four 2′-O(2-methoxyethyl) nucleotides. In a further embodiment, the antisense oligonucleotide specifically hybridizes to and reduces expression of GCCR. In a further embodiment, at least one internucleoside linkage is a phosphorothioate linkage. In a further embodiment, at least one cytosine is a 5-methylcytosine.

In a particular embodiment, the antisense oligonucleotide has the nucleobase sequence of SEQ ID: 33, wherein the antisense oligonucleotide has a 14-deoxynucleotide region flanked on its 5′ and 3′ ends with three 2′-O(2-methoxyethyl) nucleotides. In a further embodiment, the antisense oligonucleotide specifically hybridizes to and reduces expression of GCCR. In a further embodiment, at least one internucleoside linkage is a phosphorothioate linkage. In a further embodiment, least one cytosine is a 5-methylcytosine.

In a particular embodiment, the antisense oligonucleotide has the nucleobase sequence of SEQ ID: 33, wherein the antisense oligonucleotide has a 16-deoxynucleotide region flanked on its 5′ and 3′ ends with two 2′-O(2-methoxyethyl) nucleotides. In a further embodiment, the antisense oligonucleotide specifically hybridizes to and reduces expression of GCCR. In a further embodiment, at least one internucleoside linkage is a phosphorothioate linkage. In a further embodiment, at least one cytosine is a 5-methylcytosine.

In a particular embodiment, the antisense oligonucleotide has the nucleobase sequence of SEQ ID: 33, wherein the antisense oligonucleotide has a 17-deoxynucleotide region flanked on its 5′ and 3′ ends with one or two 2′-O(2-methoxyethyl) nucleotides. In a further embodiment, the antisense oligonucleotide specifically hybridizes to and reduces expression of GCCR. In a further embodiment, at least one internucleoside linkage is a phosphorothioate linkage. In a further embodiment, at least one cytosine is a 5-methylcytosine.

In a particular embodiment, the antisense oligonucleotide has the nucleobase sequence of SEQ ID: 33, wherein the antisense oligonucleotide has a 18-deoxynucleotide region flanked on its 5′ and 3′ ends with one 2′-O(2-methoxyethyl) nucleotides. In a further embodiment, the antisense oligonucleotide specifically hybridizes to and reduces expression of GCCR. In a further embodiment, at least one internucleoside linkage is a phosphorothioate linkage. In a further embodiment, at least one cytosine is a 5-methylcytosine.

The present invention provides antisense oligonucleotides comprising the nucleobase sequence of SEQ ID NO: 45. In one embodiment, the oligonucleotides of the invention comprise at least an 8-nucleobase portion of the nucleobase sequence of SEQ ID NO: 45.

In other embodiments, the present invention includes antisense oligonucleotides having the nucleobase sequence of SEQ ID NO: 45, wherein the antisense oligonucleotide is characterized by a 12-deoxynucleotide region flanked on its 5′ and 3′ ends with four 2′-O-(2-methoxyethyl) nucleotides, a 14-deoxynucleotide region flanked on its 5′ and 3′ ends with three 2′-O-(2-methoxyethyl) nucleotides, a 16-deoxynucleotide region flanked on its 5′ and 3′ ends with two 2′-O-(2-methoxyethyl) nucleotides, a 17-deoxynucleotide region flanked on its 5′ and 3′ ends with one or two 2′-O-(2-methoxyethyl) nucleotides, or an 18-deoxynucleotide region flanked on its 5′ and 3′ ends with one 2′-O-(2-methoxyethyl) nucleotides.

In a particular embodiment, the antisense oligonucleotides have the nucleobase sequence of SEQ ID: 45, wherein the antisense oligonucleotides have a 12-deoxynucleotide region flanked on its 5′ and 3′ ends with four 2′-O(2-methoxyethyl) nucleotides. In a further embodiment, the antisense oligonucleotide specifically hybridizes to and reduces expression of GCCR. In a further embodiment, at least one internucleoside linkage is a phosphorothioate linkage. In a further embodiment, at least one cytosine is a 5-methylcytosine.

In a particular embodiment, the antisense oligonucleotide has the nucleobase sequence of SEQ ID: 45, wherein the antisense oligonucleotide has a 14-deoxynucleotide region flanked on its 5′ and 3′ ends with three 2′-O(2-methoxyethyl) nucleotides. In a further embodiment, the antisense oligonucleotide specifically hybridizes to and reduces expression of GCCR. In a further embodiment, at least one internucleoside linkage is a phosphorothioate linkage. In a further embodiment, least one cytosine is a 5-methylcytosine.

In a particular embodiment, the antisense oligonucleotide has the nucleobase sequence of SEQ ID: 45, wherein the antisense oligonucleotide has a 16-deoxynucleotide region flanked on its 5′ and 3′ ends with two 2′-O(2-methoxyethyl) nucleotides. In a further embodiment, the antisense oligonucleotide specifically hybridizes to and reduces expression of GCCR. In a further embodiment, at least one internucleoside linkage is a phosphorothioate linkage. In a further embodiment, at least one cytosine is a 5-methylcytosine.

In a particular embodiment, the antisense oligonucleotide has the nucleobase sequence of SEQ ID: 45, wherein the antisense oligonucleotide has a 17-deoxynucleotide region flanked on its 5′ and 3′ ends with one or two 2′-O(2-methoxyethyl) nucleotides. In a further embodiment, the antisense oligonucleotide specifically hybridizes to and reduces expression of GCCR. In a further embodiment, at least one internucleoside linkage is a phosphorothioate linkage. In a further embodiment, at least one cytosine is a 5-methylcytosine.

In a particular embodiment, the antisense oligonucleotide has the nucleobase sequence of SEQ ID: 45, wherein the antisense oligonucleotide has a 18-deoxynucleotide region flanked on its 5′ and 3′ ends with one 2′-O(2-methoxyethyl) nucleotides. In a further embodiment, the antisense oligonucleotide specifically hybridizes to and reduces expression of GCCR. In a further embodiment, at least one internucleoside linkage is a phosphorothioate linkage. In a further embodiment, at least one cytosine is a 5-methylcytosine.

Also contemplated herein is a pharmaceutical composition comprising an antisense oligonucleotide of the invention and optionally a pharmaceutically acceptable carrier, diluent, enhancer or excipient. The compounds of the invention can also be used in the manufacture of a medicament for the treatment of diseases and disorders related to glucocorticoid activity mediated by GCCR.

Embodiments of the present invention include methods of reducing the expression of GCCR in tissues or cells comprising contacting said cells or tissues with a pharmaceutical composition or an antisense oligonucleotide of the invention, methods of decreasing blood glucose levels, blood triglyceride levels, or blood cholesterol levels in an animal comprising administering to said animal a pharmaceutical composition of the invention. Blood levels may be plasma or serum levels. Also contemplated are methods of increasing insulin sensitivity, methods of decreasing liver triglyceride levels, and methods of inhibiting hepatic glucose output in an animal comprising administering to said animal a pharmaceutical composition of the invention. Increased insulin sensitivity may be indicated by a decrease in circulating insulin levels. Another aspect of the present invention is a method of reducing body fat mass in an animal.

Other embodiments of the present invention include methods of treating an animal having a disease or condition associated with glucocorticoid receptor expression comprising administering to said animal a therapeutically or prophylactically effective amount of an antisense oligonucleotide of the invention. The disease or condition may be a metabolic disease or condition. In some embodiments, the metabolic disease or condition is diabetes, obesity, metabolic syndrome X, hyperglycemia, hyperlipidemia, or insulin resistance. In some embodiments, the disease is Type 2 diabetes. In some embodiments, the obesity is diet-induced. In some embodiments the hyperlipidemia is associated with elevated blood lipid levels. Lipids include cholesterol and triglycerides. In one embodiment, the condition is liver steatosis. In some embodiments, the steatosis is steatohepatitis or non-alcoholic steatohepatitis.

Also provided are methods of preventing or delaying the onset of elevated blood glucose or blood lipid levels in an animal.

Compounds of the invention can be used to modulate the expression of GCCR in an animal in need thereof, such as a human. In one non-limiting embodiment, the methods comprise the step of administering to said animal an effective amount of an antisense compound that reduces expression of GCCR. In one embodiment, the antisense compounds of the present invention effectively reduce the levels or function of GCCR RNA. Because reduction in GCCR mRNA levels can lead to alteration in GCCR protein products of expression as well, such resultant alterations can also be measured. Antisense compounds of the present invention that effectively reduce the levels or function of an GCCR RNA or protein products of expression is considered an active antisense compound. In one embodiment, the antisense compounds of the invention reduce the expression of GCCR causing a reduction of RNA by at least 10%, by at least 20%, by at least 25%, by at least 30%, by at least 40%, by at least 50%, by at least 60%, by at least 70%, by at least 75%, by at least 80%, by at least 85%, by at least 90%, by at least 95%, by at least 98%, by at least 99%, or by 100% as measured by an exemplified assay herein.

### Antisense Mechanisms

“Antisense mechanisms” are all those involving hybridization of a compound with target nucleic acid, wherein the outcome or effect of the hybridization is either target degradation or target occupancy with concomitant stalling of the cellular machinery involving, for example, transcription or splicing.

### Targets

As used herein, the terms “target nucleic acid” and “nucleic acid molecule encoding GCCR” have been used for convenience to encompass DNA encoding GCCR, RNA (including pre-mRNA and mRNA or portions thereof) transcribed from such DNA, and also cDNA derived from such RNA.

### Regions, Segments, and Sites

The targeting process usually also includes determination of at least one target region, segment, or site within the target nucleic acid for the antisense interaction to occur such that the desired effect, e.g., modulation of expression, will result. “Region” is defined as a portion of the target nucleic acid having at least one identifiable structure, function, or characteristic. Within regions of target nucleic acids are segments. “Segments” are defined as smaller or sub-portions of regions within a target nucleic acid. “Sites,” as used in the present invention, are defined as unique nucleobase positions within a target nucleic acid.

Once one or more target regions, segments or sites have been identified, oligomeric compounds are designed which are sufficiently complementary to the target, i.e., hybridize sufficiently well and with sufficient specificity, to give the desired effect.

### Variants

It is also known in the art that alternative RNA transcripts can be produced from the same genomic region of DNA. These alternative transcripts are generally known as “variants.” More specifically, “pre-mRNA variants” are transcripts produced from the same genomic DNA that differ from other transcripts produced from the same genomic DNA in either their start or stop position and contain both intronic and exonic sequence.

Upon excision of one or more exon or intron regions, or portions thereof during splicing, pre-mRNA variants produce smaller “mRNA variants.” Consequently, mRNA variants are processed pre-mRNA variants and each unique pre-mRNA variant must always produce a unique mRNA variant as a result of splicing. These mRNA variants are also known as “alternative splice variants.” If no splicing of the pre-mRNA variant occurs then the pre-mRNA variant is identical to the mRNA variant.

It is also known in the art that variants can be produced through the use of alternative signals to start or stop transcription and that pre-mRNAs and mRNAs can possess more that one start codon or stop codon. Variants that originate from a pre-mRNA or mRNA that use alternative start codons are known as “alternative start variants” of that pre-mRNA or mRNA. Those transcripts that use an alternative stop codon are known as “alternative stop variants” of that pre-mRNA or mRNA. One specific type of alternative stop variant is the “polyA variant” in which the multiple transcripts produced result from the alternative selection of one of the “polyA stop signals” by the transcription machinery, thereby producing transcripts that terminate at unique polyA sites. Consequently, the types of variants described herein are also suitable target nucleic acids.

### Modulation of Target Expression

“Modulation” means a perturbation of function, for example, either an increase (stimulation or induction) or a decrease (inhibition or reduction) in expression. As another example, modulation of expression can include perturbing splice site selection of pre-mRNA processing. “Expression” includes all the functions by which a gene's coded information is converted into structures present and operating in a cell. These structures include the products of transcription and translation. “Modulation of expression” means the perturbation of such functions. “Modulators” are those compounds that modulate the expression of GCCR and which comprise at least an 8-nucleobase portion which is complementary to a validated target segment.

Modulation of expression of a target nucleic acid can be achieved through alteration of any number of nucleic acid (DNA or RNA) functions. The functions of DNA to be modulated can include replication and transcription. Replication and transcription, for example, can be from an endogenous cellular template, a vector, a plasmid construct or otherwise. The functions of RNA to be modulated can include translocation functions, which include, but are not limited to, translocation of the RNA to a site of protein translation, translocation of the RNA to sites within the cell which are distant from the site of RNA synthesis, and translation of protein from the RNA. RNA processing functions that can be modulated include, but are not limited to, splicing of the RNA to yield one or more RNA species, capping of the RNA, 3′ maturation of the RNA and catalytic activity or complex formation involving the RNA which may be engaged in or facilitated by the RNA. Modulation of expression can result in the increased level of one or more nucleic acid species or the decreased level of one or more nucleic acid species, either temporally or by net steady state level. One result of such interference with target nucleic acid function is modulation of the expression of GCCR. Thus, in one embodiment modulation of expression can mean increase or decrease in target RNA or protein levels. In another embodiment modulation of expression can mean an increase or decrease of one or more RNA splice products, or a change in the ratio of two or more splice products.

### Hybridization and Complementarity

“Hybridization” means the pairing of complementary strands of oligomeric compounds. While not limited to a particular mechanism, the most common mechanism of pairing involves hydrogen bonding, which may be Watson-Crick, Hoogsteen or reversed Hoogsteen hydrogen bonding, between complementary nucleoside or nucleotide bases (nucleobases) of the strands of oligomeric compounds. For example, adenine and thymine are complementary nucleobases which pair through the formation of hydrogen bonds. Hybridization can occur under varying circumstances. An oligomeric compound is specifically hybridizable when there is a sufficient degree of complementarity to avoid non-specific binding of the oligomeric compound to non-target nucleic acid sequences under conditions in which specific binding is desired, i.e., under physiological conditions in the case of in vivo assays or therapeutic treatment, and under conditions in which assays are performed in the case of in vitro assays.

“Stringent hybridization conditions” or “stringent conditions” refer to conditions under which an oligomeric compound will hybridize to its target sequence, but to a minimal number of other sequences. Stringent conditions are sequence-dependent and will be different in different circumstances, and “stringent conditions” under which oligomeric compounds hybridize to a target sequence are determined by the nature and composition of the oligomeric compounds and the assays in which they are being investigated.

“Complementarity,” as used herein, refers to the capacity for precise pairing between two nucleobases on one or two oligomeric compound strands. For example, if a nucleobase at a certain position of an antisense compound is capable of hydrogen bonding with a nucleobase at a certain position of a target nucleic acid, then the position of hydrogen bonding between the oligonucleotide and the target nucleic acid is considered to be a complementary position. The oligomeric compound and the further DNA or RNA are complementary to each other when a sufficient number of complementary positions in each molecule are occupied by nucleobases which can hydrogen bond with each other. Thus, “specifically hybridizable” and “complementary” are terms which are used to indicate a sufficient degree of precise pairing or complementarity over a sufficient number of nucleobases such that stable and specific binding occurs between the oligomeric compound and a target nucleic acid.

It is understood in the art that the sequence of an oligomeric compound need not be 100% complementary to that of its target nucleic acid to be specifically hybridizable. Moreover, an oligonucleotide may hybridize over one or more segments such that intervening or adjacent segments are not involved in the hybridization event (e.g., a loop structure, mismatch or hairpin structure). The oligomeric compounds of the present invention comprise at least 70%, or at least 75%, or at least 80%, or at least 85%, or at least 90%, or at least 92%, or at least 95%, or at least 97%, or at least 98%, or at least 99% sequence complementarity to a target region within the target nucleic acid sequence to which they are targeted. For example, an oligomeric compound in which 18 of 20 nucleobases of the antisense compound are complementary to a target region, and would therefore specifically hybridize, would represent 90 percent complementarity. In this example, the remaining noncomplementary nucleobases may be clustered or interspersed with complementary nucleobases and need not be contiguous to each other or to complementary nucleobases. As such, an oligomeric compound which is 18 nucleobases in length having 4 (four) noncomplementary nucleobases which are flanked by two regions of complete complementarity with the target nucleic acid would have 77.8% overall complementarity with the target nucleic acid and would thus fall within the scope of the present invention. Percent complementarity of an oligomeric compound with a region of a target nucleic acid can be determined routinely using BLAST programs (basic local alignment search tools) and PowerBLAST programs known in the art (Altschul et al., J. Mol. Biol., 1990, 215, 403-410; Zhang and Madden, Genome Res., 1997, 7, 649-656). Percent homology, sequence identity or complementarity, can be determined by, for example, the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482-489).

### Oligomeric Compounds

The term “oligomeric compound” refers to a polymeric structure capable of hybridizing to a region of a nucleic acid molecule. This term includes oligonucleotides, oligonucleosides, oligonucleotide analogs, oligonucleotide mimetics and chimeric combinations of these. Oligomeric compounds are routinely prepared linearly but can be joined or otherwise prepared to be circular. Moreover, branched structures are known in the art. An “antisense compound” or “antisense oligomeric compound” refers to an oligomeric compound that is at least partially complementary to the region of a nucleic acid molecule to which it hybridizes and which modulates (increases or decreases) its expression. Consequently, while all antisense compounds can be said to be oligomeric compounds, not all oligomeric compounds are antisense compounds. An “antisense oligonucleotide” is an antisense compound that is a nucleic acid-based oligomer. An antisense oligonucleotide can be chemically modified. Nonlimiting examples of oligomeric compounds include primers, probes, antisense compounds, antisense oligonucleotides, external guide sequence (EGS) oligonucleotides, alternate splicers, and siRNAs. As such, these compounds can be introduced in the form of single-stranded, double-stranded, circular, branched or hairpins and can contain structural elements such as internal or terminal bulges or loops. Oligomeric double-stranded compounds can be two strands hybridized to form double-stranded compounds or a single strand with sufficient self complementarity to allow for hybridization and formation of a fully or partially double-stranded compound.

“Chimeric” oligomeric compounds or “chimeras,” in the context of this invention, are single- or double-stranded oligomeric compounds, such as oligonucleotides, which contain two or more chemically distinct regions, each comprising at least one monomer unit, i.e., a nucleotide in the case of an oligonucleotide compound.

A “gapmer” is defined as an oligomeric compound, generally an oligonucleotide, having a 2′-deoxyoligonucleotide region flanked by non-deoxyoligonucleotide segments. The central region is referred to as the “gap.” The flanking segments are referred to as “wings.” If one of the wings has zero non-deoxyoligonucleotide monomers, a “hemimer” is described.

### NAFLD

The term “nonalcoholic fatty liver disease” (NAFLD) encompasses a disease spectrum ranging from simple triglyceride accumulation in hepatocytes (hepatic steatosis) to hepatic steatosis with inflammation (steatohepatitis), fibrosis, and cirrhosis. Nonalcoholic steatohepatitis (NASH) occurs from progression of NAFLD beyond deposition of triglycerides. A second-hit capable of inducing necrosis, inflammation, and fibrosis is required for development of NASH. Candidates for the second-hit can be grouped into broad categories: factors causing an increase in oxidative stress and factors promoting expression of proinflammatory cytokines. It has been suggested that increased liver triglycerides lead to increased oxidative stress in hepatocytes of animals and humans, indicating a potential cause-and-effect relationship between hepatic triglyceride accumulation, oxidative stress, and the progression of hepatic steatosis to NASH (Browning and Horton, J. Clin. Invest., 2004, 114, 147-152). Hypertriglyceridemia and hyperfattyacidemia can cause triglyceride accumulation in peripheral tissues (Shimamura et al., Biochem. Biophys. Res. Commun., 2004, 322, 1080-1085). One embodiment of the present invention is a method of reducing lipids in the liver of an animal by administering a prophylactically or therapeutically effective amount of an oligomeric compound of the invention.

Another embodiment of the present invention is a method of treating hepatic steatosis in an animal by administering a prophylactically or therapeutically effective amount of an oligomeric compound of the invention. In some embodiments, the steatosis is steatohepatitis. In some embodiments, the steatotis is NASH.

### Chemical Modifications

### Modified and Alternate Nucleobases

The oligomeric compounds of the invention also include variants in which a different base is present at one or more of the nucleotide positions in the compound. For example, if the first nucleotide is an adenosine, variants may be produced which contain thymidine, guanosine or cytidine at this position. This may be done at any of the positions of the oligomeric compound. These compounds are then tested using the methods described herein to determine their ability to reduce expression of GCCR mRNA.

Oligomeric compounds can also include nucleobase (often referred to in the art as heterocyclic base or simply as “base”) modifications or substitutions. As used herein, “unmodified” or “natural” nucleobases include the purine bases adenine (A) and guanine (G), and the pyrimidine bases thymine (T), cytosine (C) and uracil (U). A “substitution” is the replacement of an unmodified or natural base with another unmodified or natural base. “Modified” nucleobases mean other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C≡C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified nucleobases include tricyclic pyrimidines such as phenoxazine cytidine(1H-pyrimido(5,4-b)(1,4)benzoxazin-2(3H)-one), phenothiazine cytidine (1H-pyrimido(5,4-b)(1,4)benzothiazin-2(3H)-one), G-clamps such as a substituted phenoxazine cytidine (e.g. 9-(2-aminoethoxy)-H-pyrimido(5,4-b)(1,4)benzoxazin-2(3H)-one), carbazole cytidine (2H-pyrimido(4,5-b)indol-2-one), pyridoindole cytidine (H-pyrido(3′,2′:4,5)pyrrolo[2,3-d]pyrimidin-2-one). Modified nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in U.S. Pat. No. 3,687,808, those disclosed in The Concise Encyclopedia Of Polymer Science And Engineering, pages 858-859, Kroschwitz, II., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B., ed., CRC Press, 1993. Certain of these nucleobases are known to those skilled in the art as suitable for increasing the binding affinity of the compounds of the invention. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. and are presently suitable base substitutions, even more particularly when combined with 2′-O-methoxyethyl sugar modifications. It is understood in the art that modification of the base does not entail such chemical modifications as to produce substitutions in a nucleic acid sequence.

Representative United States patents that teach the preparation of certain of the above noted modified nucleobases as well as other modified nucleobases include, but are not limited to, the above noted U.S. Pat. No. 3,687,808, as well as U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,645,985; 5,830,653; 5,763,588; 6,005,096; 5,681,941; and 5,750,692.

Oligomeric compounds of the present invention can also include polycyclic heterocyclic compounds in place of one or more of the naturally-occurring heterocyclic base moieties. A number of tricyclic heterocyclic compounds have been previously reported. These compounds are routinely used in antisense applications to increase the binding properties of the modified strand to a target strand. The most studied modifications are targeted to guanosines hence they have been termed G-clamps or cytidine analogs. Representative cytosine analogs that make 3 hydrogen bonds with a guanosine in a second strand include 1,3-diazaphenoxazine-2-one (Kurchavov, et al., Nucleosides and Nucleotides, 1997, 16, 1837-1846), 1,3-diazaphenothiazine-2-one, (Lin, K.-Y.; Jones, R. J.; Matteucci, M. J. Am. Chem. Soc. 1995, 117, 3873-3874) and 6,7,8,9-tetrafluoro-1,3-diazaphenoxazine-2-one (Wang, J.; Lin, K.-Y., Matteucci, M. Tetrahedron Lett. 1998, 39, 8385-8388). Incorporated into oligonucleotides these base modifications were shown to hybridize with complementary guanine and the latter was also shown to hybridize with adenine and to enhance helical thermal stability by extended stacking interactions (also see U.S. Pre-Grant Publications 20030207804 and 20030175906).

Further helix-stabilizing properties have been observed when a cytosine analog/substitute has an aminoethoxy moiety attached to the rigid 1,3-diazaphenoxazine-2-one scaffold (Lin, K.-Y.; Matteucci, M. J. Am. Chem. Soc. 1998, 120, 8531-8532). Binding studies demonstrated that a single incorporation could enhance the binding affinity of a model oligonucleotide to its complementary target DNA or RNA with a ΔTₘ of up to 18° C. relative to 5-methyl cytosine (dC5^{me}), which is a high affinity enhancement for a single modification. On the other hand, the gain in helical stability does not compromise the specificity of the oligonucleotides.

Further tricyclic heterocyclic compounds and methods of using them that are amenable to use in the present invention are disclosed in U.S. Pat. Nos. 6,028,183, and 6,007,992.

The enhanced binding affinity of the phenoxazine derivatives together with their uncompromised sequence specificity makes them valuable nucleobase analogs for the development of more potent antisense-based drugs. In fact, promising data have been derived from in vitro experiments demonstrating that heptanucleotides containing phenoxazine substitutions are capable to activate RNase H, enhance cellular uptake and exhibit an increased antisense activity (Lin, K-Y; Matteucci, M. J. Am. Chem. Soc. 1998, 120, 8531-8532). The activity enhancement was even more pronounced in case of G-clamp, as a single substitution was shown to significantly improve the in vitro potency of a 20mer 2′-deoxyphosphorothioate oligonucleotides (Flanagan, W. M.; Wolf, J. J.; Olson, P.; Grant, D.; Lin, K.-Y.; Wagner, R. W.; Matteucci, M. Proc. Natl. Acad. Sci. USA, 1999, 96, 3513-3518).

Further modified polycyclic heterocyclic compounds useful as heterocyclic bases are disclosed in but not limited to, the above noted U.S. Pat. No. 3,687,808, as well as U.S. Pat. Nos. 4,845,205; 5,130,302; 5,134,066; 5,175,273; 5,367,066; 5,432,272; 5,434,257; 5,457,187; 5,459,255; 5,484,908; 5,502,177; 5,525,711; 5,552,540; 5,587,469; 5,594,121, 5,596,091; 5,614,617; 5,645,985; 5,646,269; 5,750,692; 5,830,653; 5,763,588; 6,005,096; and 5,681,941, and U.S. Pre-Grant Publication 20030158403.

### Combinations

Compositions of the invention can contain two or more oligomeric compounds. In another related embodiment, compositions of the present invention can contain one or more antisense compounds, particularly oligonucleotides, targeted to a first nucleic acid and one or more additional antisense compounds targeted to a second nucleic acid target. Alternatively, compositions of the present invention can contain two or more antisense compounds targeted to different regions of the same nucleic acid target. Two or more combined compounds may be used together or sequentially.

### Combination Therapy

The compounds of the invention may be used in combination therapies, wherein an additive effect is achieved by administering one or more compounds of the invention and one or more other suitable therapeutic/prophylactic compounds to treat a condition. Suitable therapeutic/prophylactic compound(s) include, but are not limited to, glucose-lowering agents, anti-obesity agents, and lipid lowering agents. Glucose lowering agents include, but are not limited to hormones, hormone mimetics, or incretin mimetics (e.g., insulin, including inhaled insulin, GLP-1 or GLP-1 analogs such as liraglutide, or exenatide), DPP(IV) inhibitors, a sulfonylurea (e.g., acetohexamide, chlorpropamide, tolbutamide, tolazamide, glimepiride, a glipizide, glyburide or a gliclazide), a biguanide (metformin), a meglitinide (e.g., nateglinide or repaglinide), a thiazolidinedione or other PPAR-gamma agonists (e.g., pioglitazone or rosiglitazone) an alpha-glucosidase inhibitor (e.g., acarbose or miglitol), or an antisense compound not targeted to GCGR. Also included are dual PPAR-agonists (e.g., muraglitazar, being developed by Bristol-Myers Squibb, or tesaglitazar, being developed by Astra-Zeneca). Also included are other diabetes treatments in development (e.g. LAF237, being developed by Novartis; MK-0431, being developed by Merck; or rimonabant, being developed by Sanofi-Aventis). Anti-obesity agents include, but are not limited to, appetite suppressants (e.g. phentermine or Meridia™), fat absorption inhibitors such as orlistat (e.g. Xenical™), and modified forms of ciliary neurotrophic factor which inhibit hunger signals that stimulate appetite. Lipid lowering agents include, but are not limited to, bile salt sequestering resins (e.g., cholestyramine, colestipol, and colesevelam hydrochloride), HMGCoA-reductase inhibitors (e.g., lovastatin, pravastatin, atorvastatin, simvastatin, and fluvastatin), nicotinic acid, fibric acid derivatives (e.g., clofibrate, gemfibrozil, fenofibrate, bezafibrate, and ciprofibrate), probucol, neomycin, dextrothyroxine, plant-stanol esters, cholesterol absorption inhibitors (e.g., ezetimibe), CETP inhibitors (e.g. torcetrapib, and JTT-705) MTP inhibitors (eg, implitapide), inhibitors of bile acid transporters (apical sodium-dependent bile acid transporters), regulators of hepatic CYP7a, ACAT inhibitors (e.g. Avasimibe), estrogen replacement therapeutics (e.g., tamoxigen), synthetic HDL (e.g. ETC-216), anti-inflammatories (e.g., glucocorticoids), or an antisense compound not targeted to GCGR. One or more of these drugs may be combined with one or more of the antisense inhibitors of GCGR to achieve an additive therapeutic effect.

### Oligomer Synthesis

Oligomerization of modified and unmodified nucleosides can be routinely performed according to literature procedures for DNA (Protocols for Oligonucleotides and Analogs, Ed. Agrawal (1993), Humana Press) and/or RNA (Scaringe, Methods (2001), 23, 206-217. Gait et al., Applications of Chemically synthesized RNA in RNA: Protein Interactions, Ed. Smith (1998), 1-36. Gallo et al., Tetrahedron (2001), 57, 5707-5713) and US Publication No. US2005-0164271, which is herein incorporated by reference.

Oligomeric compounds of the present invention can be conveniently and routinely made through the well-known technique of solid phase synthesis. Equipment for such synthesis is sold by several vendors including, for example, Applied Biosystems (Foster City, Calif.). Any other means for such synthesis known in the art may additionally or alternatively be employed. It is well known to use similar techniques to prepare oligonucleotides such as the phosphorothioates and alkylated derivatives.

### Oligomer Purification and Analysis

Methods of oligonucleotide purification and analysis are known to those skilled in the art. Analysis methods include capillary electrophoresis (CE) and electrospray-mass spectroscopy. Such synthesis and analysis methods can be performed in multi-well plates.

### Nonlimiting Disclosure and Incorporation by Reference

While certain compounds, compositions and methods of the present invention have been described with specificity in accordance with certain embodiments, the examples herein serve only to illustrate the compounds of the invention and are not intended to limit the same. Each of the references, GENBANK® accession numbers, and the like recited in the present application is incorporated herein by reference in its entirety.

### Example 1

### Assaying Modulation of Expression

Modulation of GCCR expression can be assayed in a variety of ways known in the art.

GCCR mRNA levels can be quantitated by, e.g., Northern blot analysis, competitive polymerase chain reaction (PCR), or real-time PCR. RNA analysis can be performed on total cellular RNA or poly(A)+ mRNA by methods known in the art. Methods of RNA isolation are taught in, for example, Ausubel, F. M. et al., Current Protocols in Molecular Biology, Volume 1, pp. 4.1.1-4.2.9 and 4.5.1-4.5.3, John Wiley & Sons, Inc., 1993.

Northern blot analysis is routine in the art and is taught in, for example, Ausubel, F. M. et al., Current Protocols in Molecular Biology, Volume 1, pp. 4.2.1-4.2.9, John Wiley & Sons, Inc., 1996. Real-time quantitative (PCR) can be conveniently accomplished using the commercially available ABI PRISM™ 7700 Sequence Detection System, available from PE-Applied Biosystems, Foster City, Calif. and used according to manufacturer's instructions.

Levels of proteins encoded by GCCR can be quantitated in a variety of ways well known in the art, such as immunoprecipitation, Western blot analysis (immunoblotting), ELISA or fluorescence-activated cell sorting (FACS). Antibodies directed to a protein encoded by GCCR can be identified and obtained from a variety of sources, such as the MSRS catalog of antibodies (Aerie Corporation, Birmingham, Mich.), or can be prepared via conventional antibody generation methods. Methods for preparation of polyclonal antisera are taught in, for example, Ausubel, F. M. et al., Current Protocols in Molecular Biology, Volume 2, pp. 11.12.1-11.12.9, John Wiley & Sons, Inc., 1997. Preparation of monoclonal antibodies is taught in, for example, Ausubel, F. M. et al., Current Protocols in Molecular Biology, Volume 2, pp. 11.4.1-11.11.5, John Wiley & Sons, Inc., 1997.

Immunoprecipitation methods are standard in the art and can be found at, for example, Ausubel, F. M. et al., Current Protocols in Molecular Biology, Volume 2, pp. 10.16.1-10.16.11, John Wiley & Sons, Inc., 1998. Western blot (immunoblot) analysis is standard in the art and can be found at, for example, Ausubel, F. M. et al., Current Protocols in Molecular Biology, Volume 2, pp. 10.8.1-10.8.21, John Wiley & Sons, Inc., 1997. Enzyme-linked immunosorbent assays (ELISA) are standard in the art and can be found at, for example, Ausubel, F. M. et al., Current Protocols in Molecular Biology, Volume 2, pp. 11.2.1-11.2.22, John Wiley & Sons, Inc., 1991.

The effect of oligomeric compounds of the present invention on target nucleic acid expression can be tested in any of a variety of cell types provided that the target nucleic acid is present at measurable levels. The effect of oligomeric compounds of the present invention on target nucleic acid expression can be routinely determined using, for example, PCR or Northern blot analysis. Cell lines are derived from both normal tissues and cell types and from cells associated with various disorders (e.g. hyperproliferative disorders). Cell lines derived from multiple tissues and species can be obtained from American Type Culture Collection (ATCC, Manassas, Va.), the Japanese Cancer Research Resources Bank (Tokyo, Japan), or the Centre for Applied Microbiology and Research (Wiltshire, United Kingdom).

Primary cells, or those cells which are isolated from an animal and not subjected to continuous culture, can be prepared according to methods known in the art or obtained from various commercial suppliers. Additionally, primary cells include those obtained from donor human subjects in a clinical setting (i.e. blood donors, surgical patients).

### Cell Types

The effects of oligomeric compounds on target nucleic acid expression were tested in HepG2 cells and in primary rat hepatocytes.

HepG2 Cells:

The human hepatoblastoma cell line HepG2 was obtained from the American Type Culture Collection (Manassas, Va.). HepG2 cells were routinely cultured in Eagle's MEM supplemented with 10% fetal bovine serum, 1 mM non-essential amino acids, and 1 mM sodium pyruvate (Invitrogen Life Technologies, Carlsbad, Calif.). Cells were routinely passaged by trypsinization and dilution when they reached approximately 90% confluence. Multiwell culture plates are prepared for cell culture by coating with a 1:100 dilution of type 1 rat tail collagen (BD Biosciences, Bedford, Mass.) in phosphate-buffered saline. The collagen-containing plates were incubated at 37° C. for approximately 1 hour, after which the collagen was removed and the wells were washed twice with phosphate-buffered saline. Cells were seeded into 96-well plates (Falcon-Primaria #353872, BD Biosciences, Bedford, Mass.) at a density of approximately 8,000 cells/well for use in oligomeric compound transfection experiments.

Primary Rat Hepatocytes:

Primary rat hepatocytes are prepared from Sprague-Dawley rats purchased from Charles River Labs (Wilmington, Mass.) and are routinely cultured in DMEM, high glucose (Invitrogen Life Technologies, Carlsbad, Calif.) supplemented with 10% fetal bovine serum (Invitrogen Life Technologies, Carlsbad, Calif.), 100 units per mL penicillin, and 100 μg/mL streptomycin (Invitrogen Life Technologies, Carlsbad, Calif.). Cells are seeded into 96-well plates (Falcon-Primaria #353872, BD Biosciences, Bedford, Mass.) at a density of approximately 4,000-6,000 cells/well treatment with the oligomeric compounds of the invention.

Treatment with Oligomeric Compounds

When cells reached appropriate confluency, they were treated with oligonucleotide using a transfection method as described. Other suitable transfection reagents known in the art include, but are not limited to, LIPOFECTAMINE™, OLIGOFECTAMINE™, and FUGENE™. Other suitable transfection methods known in the art include, but are not limited to, electroporation.

### LIPOFECTIN™

When cells reach 65-75% confluency, they are treated with oligonucleotide.

Oligonucleotide is mixed with LIPOFECTIN™ Invitrogen Life Technologies, Carlsbad, Calif.) in Opti-MEMT™ reduced serum medium (Invitrogen Life Technologies, Carlsbad, Calif.) to achieve the desired concentration of oligonucleotide and a LIPOFECTIN™ concentration of 2.5 or 3 μg/mL per 100 nM oligonucleotide. This transfection mixture is incubated at room temperature for approximately 0.5 hours. For cells grown in 96-well plates, wells are washed once with 100 μL OPTI-MEM™-1 and then treated with 130 μL of the transfection mixture. Cells grown in 24-well plates or other standard tissue culture plates are treated similarly, using appropriate volumes of medium and oligonucleotide. Cells are treated and data are obtained in duplicate or triplicate. After approximately 4-7 hours of treatment at 37° C., the medium containing the transfection mixture is replaced with fresh culture medium. Cells are harvested 16-24 hours after oligonucleotide treatment.

### CYTOFECTIN™

When cells reach 65-75% confluency, they are treated with oligonucleotide. Oligonucleotide is mixed with CYTOFECTIN™ (Gene Therapy Systems, San Diego, Calif.) in OPTI-MEM™-1 reduced serum medium (Invitrogen Life Technologies, Carlsbad, Calif.) to achieve the desired concentration of oligonucleotide and a CYTOFECTIN™ concentration of 2 or 4 μg/mL per 100 nM oligonucleotide. This transfection mixture is incubated at room temperature for approximately 0.5 hours. For cells grown in 96-well plates, wells are washed once with 100 μL OPTI-MEM™ and then treated with 130 μL of the transfection mixture. Cells grown in 24-well plates or other standard tissue culture plates are treated similarly, using appropriate volumes of medium and oligonucleotide. Cells are treated and data are obtained in duplicate or triplicate. After approximately 4-7 hours of treatment at 37° C., the medium containing the transfection mixture is replaced with fresh culture medium. Cells are harvested 16-24 hours after oligonucleotide treatment.

### Control Oligonucleotides

Control oligonucleotides are used to determine the optimal oligomeric compound concentration for a particular cell line. Furthermore, when oligomeric compounds of the invention are tested in oligomeric compound screening experiments or phenotypic assays, control oligonucleotides are tested in parallel with compounds of the invention. In some embodiments, the control oligonucleotides are used as negative control oligonucleotides, i.e., as a means for measuring the absence of an effect on gene expression or phenotype. In alternative embodiments, control oligonucleotides are used as positive control oligonucleotides, i.e., as oligonucleotides known to affect gene expression or phenotype. Control oligonucleotides are shown in Table 2. “Target Name” indicates the gene to which the oligonucleotide is targeted. “Species of Target” indicates species in which the oligonucleotide is perfectly complementary to the target mRNA. “Motif” is indicative of chemically distinct regions comprising the oligonucleotide. Certain compounds in Table 2 are chimeric oligonucleotides, composed of a central “gap” region consisting of 2′-deoxynucleotides, which is flanked on both sides (5′ and 3′) by “wings”. The wings are composed of 2′-O-(2-methoxyethyl) nucleotides, also known as 2′-MOE nucleotides. The “motif” of each gapmer oligonucleotide is illustrated in Table 2 and indicates the number of nucleotides in each gap region and wing, for example, “5-10-5” indicates a gapmer having a 10-nucleotide gap region flanked by 5-nucleotide wings. ISIS 29848 is a mixture of randomized oligomeric compound; its sequence is shown in Table 2, where N can be A, T, C or G. The internucleoside (backbone) linkages are phosphorothioate throughout the oligonucleotides in Table 2. Unmodified cytosines are indicated by “C” in the nucleotide sequence; all other cytosines are 5-methylcytosines.

TABLE 2

Control oligonucleotides for cell line testing, oligomeric
compound screening and phenotypic assays

| | | | | | |
|---|---|---|---|---|---|
| | | | | | SEQ |
| | | | | | ID |
| ISIS # | Target Name | Species of Target | Sequence (5′ to 3′) | Motif | NO |

| | | | | | |
|---|---|---|---|---|---|
| 113131 | CD86 | Human | CGTGTGTCTGTGCTAGTCCC | 5-10-5 | 4 |
| 289865 | forkhead box O1A | Human | GGCAACGTGAACAGGTCCAA | 5-10-5 | 5 |
| | (rhabdomyosarcoma) | | | | |
| 25237 | integrin beta 3 | Human | GCCCATTGCTGGACATGC | 4-10-4 | 6 |
| 196103 | integrin beta 3 | Human | AGCCCATTGCTGGACATGCA | 5-10-5 | 7 |
| 148715 | Jagged 2 | Human; Mouse; | TTGTCCCAGTCCCAGGCCTC | 5-10-5 | 8 |
| | | Rat | | | |
| 18076 | Jun N-Terminal | Human | CTTTC^{u}CGTTGGA^{u}C^{u}CCCTGGG | 5-9-6 | 9 |
| | Kinase-1 | | | | |
| 18078 | Jun N-Terminal | Human | GTGCG^{u}CG^{u}CGAG^{u}C^{u}C^{u}CGAAATC | 5-9-6 | 10 |
| | Kinase-2 | | | | |
| 183881 | kinesin-like 1 | Human | ATCCAAGTGCTACTGTAGTA | 5-10-5 | 11 |
| 29848 | none | none | NNNNNNNNNNNNNNNNNNNN | 5-10-5 | 12 |
| 226844 | Notch (Drosophila) | Human; Mouse | GCCCTCCATGCTGGCACAGG | 5-10-5 | 13 |
| | homolog 1 | | | | |
| 105990 | Peroxisome | Human | AGCAAAAGATCAATCCGTTA | 5-10-5 | 14 |
| | proliferator-activated | | | | |
| | receptor gamma | | | | |
| 336806 | Raf kinase C | Human | TACAGAAGGCTGGGCCTTGA | 5-10-5 | 15 |
| 15770 | Raf kinase C | Mouse; Murine | ATGCATT^{u}CTG^{u}C^{u}C^{u}C^{u}C^{u}CAAGGA | 5-10-5 | 16 |
| | | sarcoma virus; Rat | | | |

The concentration of oligonucleotide used varies from cell line to cell line. To determine the optimal oligonucleotide concentration for a particular cell line, the cells are treated with a positive control oligonucleotide at a range of concentrations. Positive controls are shown in Table 2. For example, for human and non-human primate cells, the positive control oligonucleotide may be selected from ISIS 336806, or ISIS 18078. For mouse or rat cells the positive control oligonucleotide may be, for example, ISIS 15770. The concentration of positive control oligonucleotide that results in 80% reduction of the target mRNA, for example, rat Raf kinase C for ISIS 15770, is then utilized as the screening concentration for new oligonucleotides in subsequent experiments for that cell line. If 80% reduction is not achieved, the lowest concentration of positive control oligonucleotide that results in 60% reduction of the target mRNA is then utilized as the oligonucleotide screening concentration in subsequent experiments for that cell line. If 60% reduction is not achieved, that particular cell line is deemed as unsuitable for oligonucleotide transfection experiments. The concentrations of antisense oligonucleotides used herein are from 50 nM to 300 nM when the antisense oligonucleotide is transfected using a liposome reagent and 1 μM to 40 μM when the antisense oligonucleotide is transfected by electroporation.

### Example 2

### Real-Time Quantitative PCR Analysis of GCCR mRNA Levels

Quantitation of GCCR mRNA levels was accomplished by real-time quantitative PCR using the ABI PRISM™ 7600, 7700, or 7900 Sequence Detection System (PE-Applied Biosystems, Foster City, Calif.) according to manufacturer's instructions.

Gene target quantities obtained by RT, real-time PCR were normalized using either the expression level of GAPDH, a gene whose expression is constant, or by quantifying total RNA using RiboGreen™ (Molecular Probes, Inc. Eugene, Oreg.). Total RNA was quantified using RiboGreen™ RNA quantification reagent (Molecular Probes, Inc. Eugene, Oreg.). 170 μL of RiboGreen™ working reagent (RiboGreen™ reagent diluted 1:350 in 10 mM Tris-HCl, 1 mM EDTA, pH 7.5) was pipetted into a 96-well plate containing 30 μL purified cellular RNA. The plate was read in a CytoFluor 4000 (PE Applied Biosystems) with excitation at 485 nm and emission at 530 nm.

GAPDH expression was quantified by RT, real-time PCR, either simultaneously with the quantification of the target or separately. For measurement simultaneous with measurement of target levels, primer-probe sets specific to the target gene being measured were evaluated for their ability to be “multiplexed” with a GAPDH amplification reaction prior to quantitative PCR analysis. Multiplexing refers to the detection of multiple DNA species, in this case the target and endogenous GAPDH control, in a single tube, which requires that the primer-probe set for GAPDH does not interfere with amplification of the target.

Probes and primers for use in real-time PCR were designed to hybridize to target-specific sequences. Methods of primer and probe design are known in the art. Design of primers and probes for use in real-time PCR can be carried out using commercially available software, for example Primer Express®, PE Applied Biosystems, Foster City, Calif. The primers and probes and the target nucleic acid sequences to which they hybridize are presented in Table 4. The target-specific PCR probes have FAM covalently linked to the 5′ end and TAMRA or MGB covalently linked to the 3′ end, where FAM is the fluorescent dye and TAMRA or MGB is the quencher dye.

After isolation, the RNA is subjected to sequential reverse transcriptase (RT) reaction and real-time PCR, both of which are performed in the same well. RT and PCR reagents were obtained from Invitrogen Life Technologies (Carlsbad, Calif.). RT, real-time PCR was carried out in the same by adding 20 μL PCR cocktail (2.5×PCR buffer minus MgCl₂, 6.6 mM MgCl₂, 375 μM each of dATP, dCTP, dCTP and dGTP, 375 nM each of forward primer and reverse primer, 125 nM of probe, 4 Units RNAse inhibitor, 1.25 Units PLATINUM® Taq, 5 Units MuLV reverse transcriptase, and 2.5×ROX dye) to 96-well plates containing 30 μL total RNA solution (20-200 ng). The RT reaction was carried out by incubation for 30 minutes at 48° C. Following a 10 minute incubation at 95° C. to activate the PLATINUM® Taq, 40 cycles of a two-step PCR protocol were carried out: 95° C. for 15 seconds (denaturation) followed by 60° C. for 1.5 minutes (annealing/extension).

Compounds of the invention were evaluated for their effect on human target mRNA levels by quantitative real-time PCR as described in other examples herein, using a primer-probe set designed to hybridize to human GCCR. For example:

(incorporated herein as SEQ ID NO: 17)

Forward primer: TTGACATTTTGCAGGATTTGGA

(incorporated herein as SEQ ID NO: 18)

Reverse primer: CCAAGGACTCTCATTCGTCTCTTT

And the PCR probe:

(incorporated herein as SEQ ID NO: 19)

FAM-TTTCTTCTGGGTCCCC-MGB,

where FAM is the fluorescent dye and MGB is a non-fluorescent quencher dye.

Compounds of the invention were evaluated for their effect on rat target mRNA levels by quantitative real-time PCR as described in other examples herein, using a primer-probe set designed to hybridize to rat GCCR. For example:

(incorporated herein as SEQ ID NO: 20)

Forward primer: AAACAATAGTTCCTGCAGCATTACC

(incorporated herein as SEQ ID NO: 21)

Reverse primer: CATACAACACCTCGGGTTCAATC

And the PCR probe:

(incorporated herein as SEQ ID NO: 22)

FAM-ACCCCTACCTTGGTGTCACTGCT-TAMRA,

where FAM is the fluorescent dye and TAMRA is the quencher dye.

Compounds of the invention can be evaluated for their effect on mouse target mRNA levels by quantitative real-time PCR as described in other examples herein, using a primer-probe set designed to hybridize to mouse GCCR. For example:

(incorporated herein as SEQ ID NO: 23)

Forward primer: GACATCTTGCAGGATTTGGAGTT

(incorporated herein as SEQ ID NO: 24)

Reverse primer: AACAGGTCTGACCTCCAAGGACT

And the PCR probe:

(incorporated herein as SEQ ID NO: 25)

FAM-CGGGTCCCCAGGTAAAGAGACAAACGA-TAMRA,

where FAM is the fluorescent dye and TAMRA is the quencher dye.

### Example 3

### Antisense Inhibition of Human GCCR Expression by 5-10-5 Gapmers

A series of oligomeric compounds was designed to target different regions of human GCCR, using published sequences cited in Table 1. The compounds are shown in Table 3. All compounds in Table 3 are chimeric oligonucleotides (“gapmers”) 20 nucleotides in length, composed of a central “gap” region consisting of 10 2′-deoxynucleotides, which is flanked on both sides (5′ and 3′) by five-nucleotide “wings”. The wings are composed of 2′-O-(2-methoxyethyl) nucleotides, also known as 2′-MOE nucleotides. The internucleoside (backbone) linkages are phosphorothioate throughout the oligonucleotide. All cytidine residues are 5-methylcytidines. Shown in Table 3 is the sequence of the oligonucleotide, and the target site which is the first (5′ most) position on the target sequence to which the compound binds. The compounds were analyzed for their effect on gene target mRNA levels by quantitative real-time PCR as described in other examples herein, using a primer-probe set designed to hybridize to human GCCR.

Data are averages from three experiments in which HepG2 cells were treated with 50 nM of the disclosed oligomeric compounds using LIPOFECTIN™. A reduction in expression is expressed as percent inhibition in Table 3. If present, “N.D.” indicates “not determined”. The target regions to which these oligomeric compounds are inhibitory are herein referred to as “validated target segments.”

TABLE 3
Inhibition of human GCCR mRNA levels by 5-10-5 gapmers

| | Target | | | | |
|---|---|---|---|---|---|
| ISIS No of | SEQ ID | Target | | % Inhib | SEQ ID |
| 5-10-5 | NO | Site | Sequence | w/5-10-5 | NO |

| | | | | | |
|---|---|---|---|---|---|
| 361132 | 1 | 394 | TCTGTCTCTCCCATATACAG | 65 | 26 |
| 361133 | 1 | 398 | TGTTTCTGTCTCTCCCATAT | 56 | 27 |
| 361134 | 1 | 402 | CTTTTGTTTCTGTCTCTCCC | 60 | 28 |
| 361135 | 1 | 406 | ATCACTTTTGTTTCTGTCTC | 80 | 29 |
| 180272 | 1 | 497 | GTTTGCAATGCTTTCTTCCA | 74 | 30 |
| 345188 | 1 | 501 | TGAGGTTTGCAATGCTTTCT | 71 | 31 |
| 361136 | 1 | 505 | CTATTGAGGTTTGCAATGCT | 10 | 32 |
| 361137 | 1 | 509 | CGACCTATTGAGGTTTGCAA | 80 | 33 |
| 180274 | 1 | 514 | CTGGTCGACCTATTGAGGTT | 68 | 34 |
| 180275 | 1 | 672 | CTGTGGTATACAATTTCACA | 44 | 35 |
| 180276 | 1 | 679 | CTTTGGTCTGTGGTATACAA | 78 | 36 |
| 345198 | 1 | 689 | GTCAAAGGTGCTTTGGTCTG | 79 | 37 |
| 180279 | 1 | 877 | GGTTTAGTGTCCGGTAAAAT | 60 | 38 |
| 361138 | 1 | 954 | CTTTTTCTGTTTTCACTTGG | 70 | 39 |
| 180280 | 1 | 1000 | TTCTCTTGCTTAATTACCCC | 77 | 40 |
| 345218 | 1 | 1004 | CAGTTTCTCTTGCTTAATTA | 67 | 41 |
| 180281 | 1 | 1007 | GCCCAGTTTCTCTTGCTTAA | 74 | 42 |
| 361139 | 1 | 1058 | TTTATTACCAATTATATTTG | 0 | 43 |
| 361140 | 1 | 1062 | ACATTTTATTACCAATTATA | 35 | 44 |
| 361141 | 1 | 1066 | GCAGACATTTTATTACCAAT | 78 | 45 |
| 361142 | 1 | 1070 | AATGGCAGACATTTTATTAC | 40 | 46 |
| 361143 | 1 | 1074 | CAGAAATGGCAGACATTTTA | 63 | 47 |
| 361144 | 1 | 1078 | TGAACAGAAATGGCAGACAT | 61 | 48 |
| 180283 | 1 | 1081 | CCATGAACAGAAATGGCAGA | 69 | 49 |
| 361145 | 1 | 1085 | CACACCATGAACAGAAATGG | 30 | 50 |
| 361146 | 1 | 1089 | TACTCACACCATGAACAGAA | 60 | 51 |
| 361147 | 1 | 1093 | GAGGTACTCACACCATGAAC | 71 | 52 |
| 361148 | 1 | 1097 | TCCAGAGGTACTCACACCAT | 75 | 53 |
| 361149 | 1 | 1101 | GTCCTCCAGAGGTACTCACA | 69 | 54 |
| 361150 | 1 | 1105 | ATCTGTCCTCCAGAGGTACT | 53 | 55 |
| 361151 | 1 | 1109 | GTACATCTGTCCTCCAGAGG | 75 | 56 |
| 361152 | 1 | 1113 | AGTGGTACATCTGTCCTCCA | 62 | 57 |
| 361153 | 1 | 1117 | TCATAGTGGTACATCTGTCC | 52 | 58 |
| 361154 | 1 | 1121 | CATGTCATAGTGGTACATCT | 57 | 59 |
| 361155 | 1 | 1125 | TATTCATGTCATAGTGGTAC | 41 | 60 |
| 361156 | 1 | 1129 | GCTGTATTCATGTCATAGTG | 67 | 61 |
| 361157 | 1 | 1133 | GGATGCTGTATTCATGTCAT | 67 | 62 |
| 361158 | 1 | 1137 | AAAGGGATGCTGTATTCATG | 45 | 63 |
| 180288 | 1 | 1141 | TGAGAAAGGGATGCTGTATT | 62 | 64 |
| 180289 | 1 | 1181 | TGGTGGAATGACATTAAAAA | 54 | 65 |
| 361159 | 1 | 1185 | GAATTGGTGGAATGACATTA | 24 | 66 |
| 361160 | 1 | 1324 | GAGCTTACATCTGGTCTCAT | 59 | 67 |
| 361161 | 1 | 1328 | AGGAGAGCTTACATCTGGTC | 65 | 68 |
| 361162 | 1 | 1332 | ATGGAGGAGAGCTTACATCT | 18 | 69 |
| 361163 | 1 | 1336 | CTGGATGGAGGAGAGCTTAC | 50 | 70 |
| 361164 | 1 | 1339 | GAGCTGGATGGAGGAGAGCT | 49 | 71 |
| 361165 | 1 | 1468 | TGTCCTTCCACTGCTCTTTT | 61 | 72 |
| 361166 | 1 | 1472 | GTGCTGTCCTTCCACTGCTC | 65 | 73 |
| 361167 | 1 | 1476 | AATTGTGCTGTCCTTCCACT | 62 | 74 |
| 361168 | 1 | 1480 | AGGTAATTGTGCTGTCCTTC | 52 | 75 |
| 361169 | 1 | 1543 | CGGCATGCTGGGCAGTTTTT | 78 | 76 |
| 361170 | 1 | 1547 | ATAGCGGCATGCTGGGCAGT | 58 | 77 |
| 361171 | 1 | 1549 | CGATAGCGGCATGCTGGGCA | 65 | 78 |
| 361172 | 1 | 1570 | ATTCCAGCCTGAAGACATTT | 24 | 79 |
| 361173 | 1 | 1574 | GTTCATTCCAGCCTGAAGAC | 52 | 80 |
| 361174 | 1 | 1597 | TTCTTTGTTTTTCGAGCTTC | 62 | 81 |
| 361175 | 1 | 1601 | TTTTTTCTTTGTTTTTCGAG | 48 | 82 |
| 180297 | 1 | 1680 | CAGGAACTATTGTTTTGTTA | 33 | 83 |
| 361176 | 1 | 1682 | TGCAGGAACTATTGTTTTGT | 46 | 84 |
| 361177 | 1 | 1765 | GAGCTATCATATCCTGCATA | 71 | 85 |
| 361178 | 1 | 1769 | AACAGAGCTATCATATCCTG | 51 | 86 |
| 361179 | 1 | 1773 | CTGGAACAGAGCTATCATAT | 67 | 87 |
| 361180 | 1 | 1840 | TTCACTGCTGCAATCACTTG | 52 | 88 |
| 361181 | 1 | 1844 | CCATTTCACTGCTGCAATCA | 55 | 89 |
| 361182 | 1 | 1848 | TTGCCCATTTCACTGCTGCA | 70 | 90 |
| 361183 | 1 | 1999 | ATAATCAGATCAGGAGCAAA | 36 | 91 |
| 361184 | 1 | 2003 | ATTAATAATCAGATCAGGAG | 10 | 92 |
| 361185 | 1 | 2007 | GCTCATTAATAATCAGATCA | 43 | 93 |
| 361186 | 1 | 2011 | CTCTGCTCATTAATAATCAG | 0 | 94 |
| 180302 | 1 | 2015 | CATTCTCTGCTCATTAATAA | 23 | 95 |
| 180304 | 1 | 2053 | AGCATGTGTTTACATTGGTC | 73 | 96 |
| 361187 | 1 | 2119 | AAGGTTTTCATACAGAGATA | 38 | 97 |
| 361188 | 1 | 2123 | CAGTAAGGTTTTCATACAGA | 22 | 98 |
| 361189 | 1 | 2127 | GAAGCAGTAAGGTTTTCATA | 46 | 99 |
| 180307 | 1 | 2131 | GAGAGAAGCAGTAAGGTTTT | 32 | 100 |
| 361190 | 1 | 2212 | GCTTTTCCTAGCTCTTTGAT | 74 | 101 |
| 361191 | 1 | 2215 | ATGGCTTTTCCTAGCTCTTT | 68 | 102 |
| 361192 | 1 | 2347 | ATGGTCTTATCCAAAAATGT | 63 | 103 |
| 361193 | 1 | 2351 | ACTCATGGTCTTATCCAAAA | 66 | 104 |
| 361194 | 1 | 2355 | CAATACTCATGGTCTTATCC | 54 | 105 |
| 361195 | 1 | 2359 | AATTCAATACTCATGGTCTT | 69 | 106 |
| 361196 | 1 | 2383 | ATGATTTCAGCTAACATCTC | 1 | 107 |
| 180311 | 1 | 2386 | GTGATGATTTCAGCTAACAT | 59 | 108 |
| 361197 | 1 | 2407 | GAATATTTTGGTATCTGATT | 59 | 109 |
| 361198 | 1 | 2411 | ATTTGAATATTTTGGTATCT | 20 | 110 |
| 361199 | 1 | 2415 | TTCCATTTGAATATTTTGGT | 65 | 111 |
| 361200 | 1 | 2419 | ATATTTCCATTTGAATATTT | 51 | 112 |
| 361202 | 1 | 2425 | TTTTTGATATTTCCATTTGA | 20 | 113 |

The 5-10-5 gapmer oligonucleotides shown in Table 3 which reduced GCCR expression by at least 30% are preferred. The target segments to which these preferred sequences are complementary are herein referred to as “preferred target segments” and are therefore preferred for targeting by compounds of the present invention. Another aspect of the present invention is an antisense compound targeted to GCCR comprising an 8-nucleobase portion of SEQ ID NOs: 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, or 113 wherein said compound specifically hybridizes with and reduces expression of GCCR. In one embodiment the antisense compound is an antisense oligonucleotide, 20 nucleobases in length characterized by a 10-deoxynucleotide region flanked on its 5′ and 3′ ends with five 2′-O-(2-methoxyethyl) nucleotides. In one embodiment, all of the internucleoside linkages are phosphorothioate linkages. In one embodiment, all of the cytosines are 5-methylcytosines.

### Example 4

### Antisense Inhibition of Human GCCR Expression by Gap-Widened Oligonucleotides

In accordance with the present invention, gap-widened oligonucleotides having the same sequences as the compounds described in Table 4 were also tested. All compounds in Table 4 are chimeric oligonucleotides (“gapmers”) 20 nucleotides in length, composed of a central “gap” region consisting of 16 2′-deoxynucleotides, which is flanked on both sides (5′ and 3′) by two-nucleotide “wings”. The wings are composed of 2′-O-(2-methoxyethyl) nucleotides, also known as 2′-MOE nucleotides. The internucleoside (backbone) linkages are phosphorothioate throughout the oligonucleotide. All cytidine residues are 5-methylcytidines. Shown in Table 4 is the sequence of the oligonucleotide, and the target site which is the first (5′ most) position on the target sequence to which the compound binds. The 2-16-2 motif compounds were analyzed for their effect on gene target mRNA levels by quantitative real-time PCR as described herein.

Data are averages from three experiments in which HepG2 cells were treated with 50 nM of the disclosed oligomeric compounds using LIPOFECTIN™. A reduction in expression is expressed as percent inhibition in Table 4. If present, “N.D.” indicates “not determined”. The target regions to which these oligomeric compounds are inhibitory are herein referred to as “validated target segments.”

TABLE 4
Inhibition of human GCCR mRNA levels by 2-16-2 gapmers

| | Target | | | | |
|---|---|---|---|---|---|
| ISIS No of | SEQ ID | Target | | % Inhib | SEQ ID |
| 2-16-2 | NO | Site | Sequence | w/ 2-16-2 | NO |

| | | | | | |
|---|---|---|---|---|---|
| 372350 | 1 | 394 | TCTGTCTCTCCCATATACAG | 69 | 26 |
| 372376 | 1 | 398 | TGTTTCTGTCTCTCCCATAT | 72 | 27 |
| 372331 | 1 | 402 | CTTTTGTTTCTGTCTCTCCC | 67 | 28 |
| 372341 | 1 | 406 | ATCACTTTTGTTTCTGTCTC | 63 | 29 |
| 352983 | 1 | 497 | GTTTGCAATGCTTTCTTCCA | 64 | 30 |
| 372365 | 1 | 501 | TGAGGTTTGCAATGCTTTCT | 69 | 31 |
| 372387 | 1 | 505 | CTATTGAGGTTTGCAATGCT | 70 | 32 |
| 372316 | 1 | 509 | CGACCTATTGAGGTTTGCAA | 73 | 33 |
| 372310 | 1 | 514 | CTGGTCGACCTATTGAGGTT | 70 | 34 |
| 372315 | 1 | 672 | CTGTGGTATACAATTTCACA | 35 | 35 |
| 372326 | 1 | 679 | CTTTGGTCTGTGGTATACAA | 54 | 36 |
| 372339 | 1 | 689 | GTCAAAGGTGCTTTGGTCTG | 81 | 37 |
| 372322 | 1 | 877 | GGTTTAGTGTCCGGTAAAAT | 78 | 38 |
| 372361 | 1 | 954 | CTTTTTCTGTTTTCACTTGG | 70 | 39 |
| 372308 | 1 | 1000 | TTCTCTTGCTTAATTACCCC | 84 | 40 |
| 372304 | 1 | 1004 | CAGTTTCTCTTGCTTAATTA | 66 | 41 |
| 352984 | 1 | 1007 | GCCCAGTTTCTCTTGCTTAA | 80 | 42 |
| 372372 | 1 | 1058 | TTTATTACCAATTATATTTG | 0 | 43 |
| 372327 | 1 | 1062 | ACATTTTATTACCAATTATA | 11 | 44 |
| 372311 | 1 | 1066 | GCAGACATTTTATTACCAAT | 65 | 45 |
| 372352 | 1 | 1070 | AATGGCAGACATTTTATTAC | 54 | 46 |
| 372337 | 1 | 1074 | CAGAAATGGCAGACATTTTA | 36 | 47 |
| 372323 | 1 | 1078 | TGAACAGAAATGGCAGACAT | 73 | 48 |
| 372347 | 1 | 1081 | CCATGAACAGAAATGGCAGA | 86 | 49 |
| 372383 | 1 | 1085 | CACACCATGAACAGAAATGG | 73 | 50 |
| 372348 | 1 | 1089 | TACTCACACCATGAACAGAA | 82 | 51 |
| 372363 | 1 | 1093 | GAGGTACTCACACCATGAAC | 47 | 52 |
| 372334 | 1 | 1097 | TCCAGAGGTACTCACACCAT | 82 | 53 |
| 372359 | 1 | 1101 | GTCCTCCAGAGGTACTCACA | 69 | 54 |
| 372344 | 1 | 1105 | ATCTGTCCTCCAGAGGTACT | 72 | 55 |
| 372307 | 1 | 1109 | GTACATCTGTCCTCCAGAGG | 74 | 56 |
| 372370 | 1 | 1113 | AGTGGTACATCTGTCCTCCA | 69 | 57 |
| 372374 | 1 | 1117 | TCATAGTGGTACATCTGTCC | 0 | 58 |
| 372355 | 1 | 1121 | CATGTCATAGTGGTACATCT | 65 | 59 |
| 372385 | 1 | 1125 | TATTCATGTCATAGTGGTAC | 18 | 60 |
| 372319 | 1 | 1129 | GCTGTATTCATGTCATAGTG | 23 | 61 |
| 372366 | 1 | 1133 | GGATGCTGTATTCATGTCAT | 37 | 62 |
| 372330 | 1 | 1137 | AAAGGGATGCTGTATTCATG | 80 | 63 |
| 372333 | 1 | 1141 | TGAGAAAGGGATGCTGTATT | 68 | 64 |
| 372358 | 1 | 1181 | TGGTGGAATGACATTAAAAA | 67 | 65 |
| 372381 | 1 | 1185 | GAATTGGTGGAATGACATTA | 30 | 66 |
| 372377 | 1 | 1324 | GAGCTTACATCTGGTCTCAT | 45 | 67 |
| 372309 | 1 | 1328 | AGGAGAGCTTACATCTGGTC | 63 | 68 |
| 372388 | 1 | 1332 | ATGGAGGAGAGCTTACATCT | 55 | 69 |
| 372321 | 1 | 1336 | CTGGATGGAGGAGAGCTTAC | 51 | 70 |
| 372312 | 1 | 1339 | GAGCTGGATGGAGGAGAGCT | 60 | 71 |
| 372324 | 1 | 1468 | TGTCCTTCCACTGCTCTTTT | 73 | 72 |
| 372332 | 1 | 1472 | GTGCTGTCCTTCCACTGCTC | 81 | 73 |
| 372335 | 1 | 1476 | AATTGTGCTGTCCTTCCACT | 42 | 74 |
| 372342 | 1 | 1480 | AGGTAATTGTGCTGTCCTTC | 100 | 75 |
| 372345 | 1 | 1543 | CGGCATGCTGGGCAGTTTTT | 82 | 76 |
| 372356 | 1 | 1547 | ATAGCGGCATGCTGGGCAGT | 73 | 77 |
| 372305 | 1 | 1549 | CGATAGCGGCATGCTGGGCA | 80 | 78 |
| 372367 | 1 | 1570 | ATTCCAGCCTGAAGACATTT | 78 | 79 |
| 372353 | 1 | 1574 | GTTCATTCCAGCCTGAAGAC | 70 | 80 |
| 372364 | 1 | 1597 | TTCTTTGTTTTTCGAGCTTC | 47 | 81 |
| 372340 | 1 | 1601 | TTTTTTCTTTGTTTTTCGAG | 100 | 82 |
| 372369 | 1 | 1680 | CAGGAACTATTGTTTTGTTA | 56 | 83 |
| 372378 | 1 | 1682 | TGCAGGAACTATTGTTTTGT | 41 | 84 |
| 372317 | 1 | 1765 | GAGCTATCATATCCTGCATA | 84 | 85 |
| 372351 | 1 | 1769 | AACAGAGCTATCATATCCTG | 69 | 86 |
| 372389 | 1 | 1773 | CTGGAACAGAGCTATCATAT | 76 | 87 |
| 372362 | 1 | 1840 | TTCACTGCTGCAATCACTTG | 64 | 88 |
| 372328 | 1 | 1844 | CCATTTCACTGCTGCAATCA | 81 | 89 |
| 372338 | 1 | 1848 | TTGCCCATTTCACTGCTGCA | 82 | 90 |
| 372349 | 1 | 1999 | ATAATCAGATCAGGAGCAAA | 10 | 91 |
| 372373 | 1 | 2003 | ATTAATAATCAGATCAGGAG | 30 | 92 |
| 372360 | 1 | 2007 | GCTCATTAATAATCAGATCA | 27 | 93 |
| 372384 | 1 | 2011 | CTCTGCTCATTAATAATCAG | 100 | 94 |
| 372380 | 1 | 2015 | CATTCTCTGCTCATTAATAA | 2 | 95 |
| 372320 | 1 | 2053 | AGCATGTGTTTACATTGGTC | 75 | 96 |
| 372371 | 1 | 2119 | AAGGTTTTCATACAGAGATA | 37 | 97 |
| 372382 | 1 | 2123 | CAGTAAGGTTTTCATACAGA | 44 | 98 |
| 372306 | 1 | 2127 | GAAGCAGTAAGGTTTTCATA | 48 | 99 |
| 372343 | 1 | 2131 | GAGAGAAGCAGTAAGGTTTT | 46 | 100 |
| 372313 | 1 | 2212 | GCTTTTCCTAGCTCTTTGAT | 66 | 101 |
| 372325 | 1 | 2215 | ATGGCTTTTCCTAGCTCTTT | 69 | 102 |
| 372336 | 1 | 2347 | ATGGTCTTATCCAAAAATGT | 65 | 103 |
| 372318 | 1 | 2351 | ACTCATGGTCTTATCCAAAA | 70 | 104 |
| 372375 | 1 | 2355 | CAATACTCATGGTCTTATCC | 85 | 105 |
| 372346 | 1 | 2359 | AATTCAATACTCATGGTCTT | 47 | 106 |
| 372386 | 1 | 2383 | ATGATTTCAGCTAACATCTC | 74 | 107 |
| 372354 | 1 | 2386 | GTGATGATTTCAGCTAACAT | 66 | 108 |
| 372357 | 1 | 2407 | GAATATTTTGGTATCTGATT | 13 | 109 |
| 372368 | 1 | 2411 | ATTTGAATATTTTGGTATCT | 0 | 110 |
| 372379 | 1 | 2415 | TTCCATTTGAATATTTTGGT | 44 | 111 |
| 372390 | 1 | 2419 | ATATTTCCATTTGAATATTT | 0 | 112 |
| 372329 | 1 | 2425 | TTTTTGATATTTCCATTTGA | 0 | 113 |

The 2-16-2 oligonucleotides shown in Table 4 which reduced GCCR expression by at least 30% are preferred. The target segments to which these preferred sequences are complementary are herein referred to as “preferred target segments” and are therefore preferred for targeting by compounds of the present invention.

Another aspect of the present invention is an antisense compound targeted to GCCR comprising an 8-nucleobase portion of SEQ ID NOs: 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, or 113 wherein said compound specifically hybridizes with and reduces expression of GCCR. In one embodiment the antisense compound is an antisense oligonucleotide, 20 nucleobases in length characterized by a 16-deoxynucleotide region flanked on its 5′ and 3′ ends with two 2′-O-(2-methoxyethyl) nucleotides. In one embodiment, all of the internucleoside linkages are phosphorothioate linkages. In one embodiment, all of the cytosines are 5-methylcytosines.

### Example 5

### Cross-Species Oligonucleotides Targeting GCCR

Some oligonucleotides described in the previous example are complementary across species and are therefore expected to reduce expression of glucocorticoid receptor across species. Shown in Table 5 is the sequence of such cross-species oligonucleotides, and the ISIS numbers of the 5-10-5 motif version and the 2-16-2 motif version of the oligonucleotide. Also indicated for each sequence is the target site which is the first (5′ most) position on the human target sequence (NM_{—}000176.1, incorporated herein as SEQ ID NO: 1) to which the compound binds. The complementarity for human, cynomolgus monkey, rat, and mouse GCCR mRNA is indicated (“yes” means perfect complementarity and “1 mm” means one mismatch from perfect complementarity).

TABLE 5
Cross-species oligonucleotides targeted to GCCR

| | | | Pos'n | |
|---|---|---|---|---|
| ISIS # of | ISIS # of | SEQ | on | |
| 5-10-5 | 2-16-2 | ID | SEQ ID | Perfect complement to: |

| | | | | | | | | |
|---|---|---|---|---|---|---|---|---|
| gapmer | gapmer | NO | Sequence | NO: 1 | Human | Monkey | Rat | Mouse |

| | | | | | | | | |
|---|---|---|---|---|---|---|---|---|
| 361137 | 372316 | 33 | cgacctattgaggtttgcaa | 509 | yes | yes | yes | yes |
| 180276 | 372326 | 36 | ctttggtctgtggtatacaa | 679 | yes | 1 mm | 1 mm | yes |
| 345198 | 372339 | 37 | gtcaaaggtgctttggtctg | 689 | yes | yes | yes | yes |
| 180304 | 372320 | 96 | agcatgtgtttacattggtc | 2053 | yes | yes | yes | yes |
| 180275 | 372315 | 35 | ctgtggtatacaatttcaca | 672 | yes | 1 mm | 1 mm | yes |
| 361141 | 372311 | 45 | gcagacattttattaccaat | 1066 | yes | yes | yes | 1 mm |
| 180281 | 352984 | 42 | gcccagtttctcttgcttaa | 1007 | yes | yes | yes | yes |
| 361151 | 372307 | 56 | gtacatctgtcctccagagg | 1109 | yes | yes | yes | yes |
| 180274 | 372310 | 34 | ctggtcgacctattgaggtt | 514 | yes | yes | yes | yes |
| 361156 | 372319 | 61 | gctgtattcatgtcatagtg | 1129 | yes | yes | yes | yes |

### Example 6

### Antisense Inhibition of Human and Rat GCCR mRNA Levels—Dose-Response Studies with 5-10-5 Gapmers

In a further embodiment of the present invention, eleven oligonucleotides were selected for additional dose-response studies. Primary rat hepatocytes were treated with 5, 10, 25, 50, 100 or 200 nM of ISIS 180274, ISIS 180275, ISIS 180276, ISIS 180281, ISIS 180304, ISIS 361137, ISIS 361141, ISIS 361151, ISIS 361156, ISIS 345198, ISIS 361137 or the negative control oligonucleotide ISIS 141923 (CCTTCCCTGAAGGTTCCTCC, incorporated herein as SEQ ID NO: 114), and mRNA levels were measured as described in other examples herein. ISIS 141923 is a 5-10-5 gapmer comprising a ten deoxynucleotide gap flanked by 2′-MOE wings and a phosphorothioate backbone. All cytosines are 5-methylcytosines. Untreated cells served as the control to which the data were normalized.

Results of these studies are shown in Table 6. Target mRNA levels were measured by real-time PCR as described herein. Data are averages from three experiments and are expressed as percent inhibition relative to untreated control.

TABLE 6
Dose-dependent inhibition of GCCR expression
in rat primary hepatocytes

| | | % Inhibition | | | | | |
|---|---|---|---|---|---|---|---|
| | | Dose of Oligonucleotide (nM) | | | | | |
| ISIS # | SEQ ID NO | 5 | 10 | 25 | 50 | 100 | 200 |
| 180274 | 34 | 16 | 33 | 29 | 65 | 84 | 89 |
| 180275 | 35 | 0 | 13 | 56 | 84 | 84 | 90 |
| 180276 | 36 | 23 | 43 | 43 | 68 | 89 | 93 |
| 180281 | 42 | 0 | 20 | 33 | 75 | 86 | 87 |
| 180304 | 96 | 42 | 51 | 47 | 75 | 86 | 91 |
| 361137 | 33 | 40 | 30 | 48 | 81 | 83 | 89 |
| 361141 | 45 | 36 | 61 | 48 | 77 | 87 | 92 |
| 361151 | 56 | 10 | 28 | 42 | 77 | 90 | 94 |
| 361156 | 61 | 22 | 47 | 46 | 66 | 84 | 92 |
| 345198 | 37 | 0 | 35 | 53 | 81 | 77 | 85 |
| 361158 | 63 | 34 | 50 | 47 | 79 | 91 | 93 |
| 141923 | 114 | 0 | 10 | 18 | 43 | 0 | 12 |

In a further embodiment of the present invention, the same oligonucleotides were tested in the human HepG2 cell line for their ability to reduce GCCR mRNA expression at the indicated doses. Untreated cells served as the control to which the data were normalized.

Results of these studies are shown in Table 7. Target mRNA levels were measured by real-time PCR as described herein. Data are averages from three experiments and are expressed as percent inhibition relative to untreated control.

TABLE 7
Dose-dependent inhibition of GCCR expression in HepG2 cells

| | | % Inhibition | | | | | |
|---|---|---|---|---|---|---|---|
| | | Dose of Oligonucleotide (nM) | | | | | |
| ISIS # | SEQ ID NO | 1 | 10 | 25 | 50 | 100 | 200 |
| 180274 | 34 | 0 | 31 | 54 | 66 | 77 | 83 |
| 180275 | 35 | 13 | 54 | 75 | 86 | 93 | 94 |
| 180276 | 36 | 26 | 77 | 87 | 92 | 94 | 98 |
| 180281 | 42 | 3 | 46 | 68 | 80 | 90 | 84 |
| 180304 | 96 | 0 | 64 | 90 | 90 | 92 | 91 |
| 361137 | 33 | 18 | 71 | 84 | 91 | 92 | 86 |
| 361141 | 45 | 1 | 49 | 81 | 85 | 73 | 78 |
| 361151 | 56 | 22 | 42 | 71 | 82 | 89 | 91 |
| 361156 | 61 | 7 | 75 | 75 | 79 | 80 | 82 |
| 345198 | 37 | 17 | 71 | 79 | 86 | 80 | 82 |
| 361158 | 63 | 11 | 35 | 78 | 80 | 82 | 77 |
| 141923 | 114 | 15 | 12 | 20 | 12 | 14 | 3 |

As shown in Table 6 and Table 7, antisense oligonucleotides targeting GCCR are effective at reducing both human and rat target mRNA levels in a dose-dependent manner.

### Example 7

### Antisense Inhibition of Rat GCCR mRNA Levels—In Vivo Dose-Response Studies with 5-10-5 Gapmers

Five of the 5-10-5 gapmer motif oligonucleotides (ISIS180281, ISIS 361137, ISIS 345198, ISIS180304, and ISIS 361141) were evaluated at various doses in rats for their ability to reduce GCCR mRNA levels in liver. Eight week-old Sprague Dawley rats were divided into treatment groups which received doses of 50, 25 or 12.5 mg/kg of one the indicated oligonucleotides via injection. Each treatment group was comprised of four animals, and was dosed twice weekly for 3 weeks. Animals injected with saline alone served as a control group. The animals were evaluated weekly for standard blood parameters (ALT/AST, cholesterol, triglycerides, and glucose). Animals were sacrificed at the end of the study and liver tissue was collected and analyzed for target reduction using real-time PCR analysis methods described herein. Results are shown in Tables 8a and 8b (separate experiments) as the percentage reduction in GCCR mRNA measured after treatment with the indicated doses of the indicated oligonucleotides.

TABLE 8a
In vivo rat screen- GCCR antisense oligonucleotides

| | % Reduction in GCCR mRNA in rat liver | | |
|---|---|---|---|
| | (compared to saline-treated controls) | | |
| Compound | 50 mg/kg | 25 mg/kg | 12.5 mg/kg |
| ISIS 180281 | 68 | 65 | 48 |
| ISIS 180304 | 52 | 34 | 0 |
| ISIS 345198 | 63 | 58 | 52 |

TABLE 8b
In vivo rat screen- GCCR antisense oligonucleotides

| | % Reduction in GCCR mRNA in rat liver | | |
|---|---|---|---|
| | (compared to saline-treated controls) | | |
| Compound | 50 mg/kg | 25 mg/kg | 12.5 mg/kg |
| ISIS 180281 | 62 | 62 | 59 |
| ISIS 361137 | 59 | 47 | 32 |
| ISIS 361141 | 61 | 49 | 22 |

The data in Tables 8a and 8b show that antisense oligonucleotides targeted to GCCR are effective at reducing expression in vivo in a dose-dependent manner ISIS 345198 (GTCAAAGGTGCTTTGGTCTG; SEQ ID NO: 37) was chosen for further evaluation in structure-activity experiments focusing on gap optimization. This compound is perfectly complementary to mouse, rat, human, monkey, rabbit and guinea pig glucocorticoid receptor RNA.

### Example 8

### Antisense Inhibition of GCCR mRNA Levels In Vivo—Gap Optimization Study

A series of oligomeric compounds were designed to target GCCR with varying sizes of the deoxynucleotide gap and 2′-MOE wings. Each of the oligonucleotides tested has the same nucleobase sequence (GTCAAAGGTGCTTTGGTCTG, incorporated herein as SEQ ID NO: 37) and therefore targets the same segment of SEQ ID NO: 1 (nucleobases 689 to 709). As shown in Example 5, this oligonucleotide is also perfectly complementary to rat GCCR.

The compounds are shown in Table 9. Plain text indicates a deoxynucleotide, and nucleobases designated with bold, underlined text are 2′-O-(2-methoxyethyl) nucleotides. Internucleoside linkages are phosphorothioate throughout, and all cytosines are 5-methylcytosines. Indicated in Table 9 is the “motif” of each compound indicative of chemically distinct regions comprising the oligonucleotide.

TABLE 9
Antisense compounds targeting rat GCCR

| ISIS | | SEQ | |
|---|---|---|---|
| Number | Chemistry | ID NO: | Motif |
| 345198 | GTCAAAGGTGCTTTGGTCTG | 37 | 5-10-5 gapmer |
| 372339 | GTCAAAGGTGCTTTGGTCTG | 37 | 2-16-2 gapmer |
| 377130 | GTCAAAGGTGCTTTGGTCTG | 37 | 3-14-3 gapmer |
| 377131 | GTCAAAGGTGCTTTGGTCTG | 37 | 4-12-4 gapmer |

Nine-week old Sprague-Dawley male rats were treated twice weekly for three weeks with doses of 50, 25, 12.5, and 6.25 mg/kg of the oligonucleotides presented in Table 9. Animals injected with saline alone served as controls. Each treatment group was comprised of four animals and animals were monitored weekly for plasma transaminases, lipids, glucose levels and body weight gain. As expected for normal animals, no substantial alterations in glucose were observed. Baseline (prior to the start of treatment) plasma cholesterol (CHOL) and triglyceride (TRIG) levels and levels measured at week 3 are shown in Table 10 in mg/dL as the average for each treatment group.

TABLE 10
Effect of oligonucleotides targeted to GCCR
on plasma lipids levels in normal rats

| | Baseline | Week 3 | Baseline | Week 3 |
|---|---|---|---|---|
| | TRIG | TRIG | CHOL | CHOL |
| Treatment | (mg/dL) | (mg/dL) | (mg/dL) | (mg/dL) |
| Saline | 78 | 70 | 77 | 62 |
| 345198, 50 mg/kg | 50 | 23 | 66 | 35 |
| 345198, 25 mg/kg | 99 | 34 | 69 | 39 |
| 345198, 12.5 mg/kg | 71 | 52 | 64 | 42 |
| 345198, 6.25 mg/kg | 139 | 99 | 78 | 58 |
| 372339, 50 mg/kg | 93 | 29 | 75 | 54 |
| 372339, 25 mg/kg | 86 | 33 | 70 | 40 |
| 372339, 12.5 mg/kg | 104 | 71 | 69 | 49 |
| 372339, 6.25 mg/kg | 103 | 102 | 71 | 56 |
| 377130, 50 mg/kg | 91 | 21 | 65 | 41 |
| 377130, 25 mg/kg | 82 | 32 | 75 | 41 |
| 377130, 12.5 mg/kg | 84 | 68 | 72 | 47 |
| 377130, 6.25 mg/kg | 76 | 67 | 70 | 52 |
| 377131, 50 mg/kg | 96 | 28 | 85 | 48 |
| 377131, 25 mg/kg | 83 | 25 | 75 | 42 |
| 377131, 12.5 mg/kg | 64 | 49 | 79 | 44 |
| 377131, 6.25 mg/kg | 119 | 110 | 75 | 60 |

As shown in Table 10, treatment with antisense oligonucleotides targeted to GCCR caused dose-dependent decreases in cholesterol and triglyceride levels. Therefore, one embodiment of the present invention is a method of decreasing blood lipid levels in an animal comprising administering to said animal a gap-widened oligonucleotide. In a preferred embodiment, the gap-widened oligonucleotide has the sequence of SEQ ID NO: 37. In other preferred embodiments, the gap-widened oligonucleotide is ISIS 372339, ISIS 377130, or ISIS 377131.

At the end of the study, animals were sacrificed, organ weights were measured, and tissues were collected for determination of target reduction and oligonucleotide concentration.

White adipose tissue was analyzed for target reduction using real-time PCR analysis methods described herein. Results are shown in Tables 11a, 11b, and 11c (separate experiments) as the percentage reduction in GCCR mRNA measured after treatment with the indicated doses of the indicated oligonucleotides. Tissues from animals treated with each gap-widened oligonucleotide were assayed for target reduction alongside tissues from animals treated with the 5-10-5 motif oligonucleotide for comparison.

TABLE 12a
In vivo reduction of GCCR levels in white
adipose tissue with 2-16-2 oligonucleotides

| | |
|---|---|
| | % Inhibition |
| Treatment | Dose of oligonucleotide (mg/kg) |

| group | 50 | 25 | 12.5 | 6.25 |
|---|---|---|---|---|
| ISIS 345198 | 56 | 26 | 17 | 7 |
| ISIS 372339 | 34 | 0 | 8 | 8 |

TABLE 11b
In vivo reduction of GCCR levels in white
adipose tissue with 3-14-3 oligonucleotides

| | |
|---|---|
| | % Inhibition |
| Treatment | Dose of oligonucleotide (mg/kg) |

| group | 50 | 25 | 12.5 | 6.25 |
|---|---|---|---|---|
| ISIS 345198 | 59 | 49 | 27 | 22 |
| ISIS 377130 | 54 | 37 | 21 | 18 |

TABLE 11c
In vivo reduction of GCCR levels in white
adipose tissue with 4-12-4 oligonucleotides

| | |
|---|---|
| | % Inhibition |
| Treatment | Dose of oligonucleotide (mg/kg) |

| group | 50 | 25 | 12.5 | 6.25 |
|---|---|---|---|---|
| ISIS 345198 | 56 | 23 | 21 | 7 |
| ISIS 377131 | 55 | 23 | 15 | 0 |

Liver tissue was also analyzed for target reduction using real-time PCR analysis methods described herein. Results are shown in Tables 12a, 12b, and 12c (separate experiments) as the percentage reduction in GCCR mRNA measured after treatment with the indicated doses of the indicated oligonucleotides. Tissues from animals treated with each gap-widened oligonucleotide were assayed for target reduction alongside tissues from animals treated with the 5-10-5 motif oligonucleotide for comparison.

TABLE 12a
In vivo reduction of GCCR levels in
liver with 2-16-2 oligonucleotides

| | |
|---|---|
| | % Inhibition |
| Treatment | Dose of oligonucleotide (mg/kg) |

| group | 50 | 25 | 12.5 | 6.25 |
|---|---|---|---|---|
| ISIS 345198 | 78 | 77 | 65 | 51 |
| ISIS 372339 | 83 | 77 | 56 | 44 |

TABLE 12b
In vivo reduction of GCCR levels in
liver with 3-14-3 oligonucleotides

| | |
|---|---|
| | % Inhibition |
| Treatment | Dose of oligonucleotide (mg/kg) |

| group | 50 | 25 | 12.5 | 6.25 |
|---|---|---|---|---|
| ISIS 345198 | 78 | 80 | 67 | 54 |
| ISIS 377130 | 87 | 78 | 68 | 43 |

TABLE 12c
In vivo reduction of GCCR levels in
liver with 4-12-4 oligonucleotides

| | |
|---|---|
| | % Inhibition |
| Treatment | Dose of oligonucleotide (mg/kg) |

| group | 50 | 25 | 12.5 | 6.25 |
|---|---|---|---|---|
| ISIS 345198 | 76 | 75 | 58 | 49 |
| ISIS 377131 | 82 | 64 | 60 | 61 |

As shown in Tables 11a, 11b, and 11c, all of the gap-widened oligonucleotides tested were effective at reducing GCCR levels in a dose-dependent manner in vivo. In addition, the gap-widened oligonucleotides show a trend toward greater potency than the 5-10-5 gapmer in the liver.

In addition, to determine effects of altering the gap size on pharmacokinetics, oligonucleotide concentration in kidney and liver were determined. Methods to determine oligonucleotide concentration in tissues are known in the art (Geary et al., Anal Biochem, 1999, 274, 241-248). Total oligonucleotide is the sum of all oligonucleotides metabolites detected in the tissue. Shown in Table 12 are the total concentration and the concentration of full length oligonucleotide (in μg/g) in the liver of animals treated with the indicated oligonucleotide at the indicated concentration.

TABLE 12
GCCR oligonucleotide concentration in rat liver

| | | | Liver | Liver |
|---|---|---|---|---|
| | | | Total | Full- |
| Treatment | Motif | Dose | oligo | length |
| ISIS 345198 | 5-10-5 | 25 mg/kg | 507 | 408 |
| | | 12.5 mg/kg | 318 | 224 |
| ISIS 372339 | 2-16-2 | 25 mg/kg | 450 | 306 |
| | | 12.5 mg/kg | 311 | 183 |
| ISIS 377130 | 3-14-3 | 25 mg/kg | 575 | 315 |
| | | 12.5 mg/kg | 350 | 212 |
| ISIS 377131 | 4-12-4 | 25 mg/kg | 584 | 424 |
| | | 12.5 mg/kg | 354 | 265 |

As shown in Table 12, the levels of full-length oligonucleotide in the liver are comparable or reduced for ISIS 372339 and ISIS 377130 as compared to ISIS 345198. Coupled with the target reduction as shown in Table 11, these data show that the enhanced potency of the gap-widened compounds is not due to enhanced accumulation of the compound in the liver. Thus, preferred oligonucleotides of the present invention include gap-widened oligonucleotides that show enhanced or comparable potency with regard to target reduction to the corresponding 5-10-5 gapmer without enhanced accumulation of the compound in a target tissue. In some embodiments, the target tissue is adipose and in some embodiments, the target tissue is liver.

What is claimed is:

1. An antisense oligonucleotide 13 to 17 nucleobases in length targeted to a nucleic acid molecule encoding GCCR and comprising at least an 8-nucleobase portion of SEQ ID NO: 37 wherein the oligonucleotide comprises a deoxynucleotide region 12, 13, 14, or 15 nucleobases in length which is flanked on its 5′ and 3′ ends with 1 to 4 2′-O-(2-methoxyethyl) nucleotides.

2. The antisense oligonucleotide of claim 1 wherein the number of nucleotides flanking the deoxynucleotide region on the 5′ and 3′ ends is the same.

3. The antisense oligonucleotide of claim 1 wherein the number of nucleotides flanking the deoxynucleotide region on the 5′ and 3′ ends is not the same.

4. The antisense oligonucleotide of claim 1 wherein at least one internucleoside linkage is a phosphorothioate linkage.

5. The antisense oligonucleotide of claim 1 wherein at least one cytosine is a 5-methylcytosine.

6. The antisense oligonucleotide of claim 1 having the nucleobase sequence of SEQ ID NO: 37.

7. The antisense oligonucleotide of claim 6 characterized by a 12-deoxynucleotide region flanked on its 5′ and 3′ ends with four 2′-0-(2-methoxyethyl) nucleotides.

8. A pharmaceutical composition comprising the antisense oligonucleotide of claim 1 and a pharmaceutically acceptable carrier, diluent, enhancer or excipient.

9. A method of reducing expression of glucocorticoid receptor in a cell or tissue comprising contacting said cell or tissue with the pharmaceutical composition of claim 54.

10. The method of claim 9 wherein the tissue is fat or liver tissue.

11. A method of treating a disease or condition mediated by glucocorticoid expression in an animal comprising contacting said animal with an effective amount of the pharmaceutical composition of claim 8.

12. The method of claim 11 wherein the disease or condition is diabetes, obesity, metabolic syndrome X, hyperglycemia, or hyperlipidemia.

13. The method of claim 11 wherein the disease is Type 2 diabetes.

14. The method of claim 11 wherein the disease is hyperlipidemia associated with elevated blood cholesterol or elevated blood triglyceride levels.

15. An oligomeric compound 13 to 17 nucleobases in length targeted to a nucleic acid molecule encoding GCCR, wherein the compound comprises a deoxynucleotide region 11-15 nucleobases in length flanked on each of its 5′ and 3′ ends with at least one 2′-O-(2-methoxyethyl) nucleotide.

16. The compound of claim 15, wherein the deoxynucleotide region is 12, 13, 14, or 15 nucleobases in length and is flanked on its 5′ and 3′ ends with 1 to 4 2′O′(2-methoxyethyl) nucleotides.

17. The compound of claim 15, wherein the compound is targeted to a target region comprising nucleotides 672 to 698, 497 to 533 or 1062 to 1100 of SEQ ID NO: 1.

18. The compound of claim 17, wherein the target region comprises nucleotides 672 to 698 and wherein the compound further comprises at least an 8-nucleobase portion of SEQ ID NO: 35, 36 or 37.

19. The compound of claim 17, wherein the target region comprises nucleotides 497 to 533 and wherein the compound further comprises at least an 8-nucleobase portion of SEQ ID NO: 30, 31, 32, 33 or 34.

20. The compound of claim 17, wherein the target region comprises nucleotides 1062 to 1100 and wherein the compound further comprises at least an 8-nucleobase portion of SEQ ID NO: 44, 45, 46, 47, 48 or 49.


SEQUENCE LISTING (114 sequences)

SEQ ID NO: 1 - DNA, 4788 bp, H. sapiens
tttttagaaaaaaaaaatatatttccctcctgctccttctgcgttcacaagctaagttgtttatctcggctgcggcgggaactgcggacggtggcgggcgagcggctcctctgccagagttgatattcactgatggactccaaagaatcattaactcctggtagagaagaaaaccccagcagtgtgcttgctcaggagaggggagatgtgatggacttctataaaaccctaagaggaggagctactgtgaaggtttctgcgtcttcaccctcactggctgtcgcttctcaatcagactccaagcagcgaagacttttggttgattttccaaaaggctcagtaagcaatgcgcagcagccagatctgtccaaagcagtttcactctcaatgggactgtatatgggagagacagaaacaaaagtgatgggaaatgacctgggattcccacagcagggccaaatcagcctttcctcgggggaaacagacttaaagcttttggaagaaagcattgcaaacctcaataggtcgaccagtgttccagagaaccccaagagttcagcatccactgctgtgtctgctgcccccacagagaaggagtttccaaaaactcactctgatgtatcttcagaacagcaacatttgaagggccagactggcaccaacggtggcaatgtgaaattgtataccacagaccaaagcacctttgacattttgcaggatttggagttttcttctgggtccccaggtaaagagacgaatgagagtccttggagatcagacctgttgatagatgaaaactgtttgctttctcctctggcgggagaagacgattcattccttttggaaggaaactcgaatgaggactgcaagcctctcattttaccggacactaaacccaaaattaaggataatggagatctggttttgtcaagccccagtaatgtaacactgccccaagtgaaaacagaaaaagaagatttcatcgaactctgcacccctggggtaattaagcaagagaaactgggcacagtttactgtcaggcaagctttcctggagcaaatataattggtaataaaatgtctgccatttctgttcatggtgtgagtacctctggaggacagatgtaccactatgacatgaatacagcatccctttctcaacagcaggatcagaagcctatttttaatgtcattccaccaattcccgttggttccgaaaattggaataggtgccaaggatctggagatgacaacttgacttctctggggactctgaacttccctggtcgaacagttttttctaatggctattcaagccccagcatgagaccagatgtaagctctcctccatccagctcctcaacagcaacaacaggaccacctcccaaactctgcctggtgtgctctgatgaagcttcaggatgtcattatggagtcttaacttgtggaagctgtaaagttttcttcaaaagagcagtggaaggacagcacaattacctatgtgctggaaggaatgattgcatcatcgataaaattcgaagaaaaaactgcccagcatgccgctatcgaaaatgtcttcaggctggaatgaacctggaagctcgaaaaacaaagaaaaaaataaaaggaattcagcaggccactacaggagtctcacaagaaacctctgaaaatcctggtaacaaaacaatagttcctgcaacgttaccacaactcacccctaccctggtgtcactgttggaggttattgaacctgaagtgttatatgcaggatatgatagctctgttccagactcaacttggaggatcatgactacgctcaacatgttaggagggcggcaagtgattgcagcagtgaaatgggcaaaggcaataccaggtttcaggaacttacacctggatgaccaaatgaccctactgcagtactcctggatgtttcttatggcatttgctctggggtggagatcatatagacaatcaagtgcaaacctgctgtgttttgctcctgatctgattattaatgagcagagaatgactctaccctgcatgtacgaccaatgtaaacacatgctgtatgtttcctctgagttacacaggcttcaggtatcttatgaagagtatctctgtatgaaaaccttactgcttctctcttcagttcctaaggacggtctgaagagccaagagctatttgatgaaattagaatgacctacatcaaagagctaggaaaagccattgtcaagagggaaggaaactccagccagaactggcagcggttttatcaactgacaaaactcttggattctatgcatgaagtggttgaaaatctccttaactattgcttccaaacatttttggataagaccatgagtattgaattccccgagatgttagctgaaatcatcaccaatcagataccaaaatattcaaatggaaatatcaaaaaacttctgtttcatcaaaagtgactgccttaataagaatggttgccttaaagaaagtcgaattaatagcttttattgtataaactatcagtttgtcctgtagaggttttgttgttttattttttattgttttcatctgttgttttgttttaaatacgcactacatgtggtttatagagggccaagacttggcaacagaagcagttgagtcgtcatcacttttcagtgatgggagagtagatggtgaaatttattagttaatatatcccagaaattagaaaccttaatatgtggacgtaatctccacagtcaaagaaggatggcacctaaaccaccagtgcccaaagtctgtgtgatgaactttctcttcatactttttttcacagttggctggatgaaattttctagactttctgttggtgtatcccccccctgtatagttaggatagcatttttgatttatgcatggaaacctgaaaaaaagtttacaagtgtatatcagaaaagggaagttgtgccttttatagctattactgtctggttttaacaatttcctttatatttagtgaactacgcttgctcattttttcttacataattttttattcaagttattgtacagctgtttaagatgggcagctagttcgtagctttcccaaataaactctaaacattaatcaatcatctgtgtgaaaatgggttggtgcttctaacctgatggcacttagctatcagaagaccacaaaaattgactcaaatctccagtattcttgtcaaaaaaaaaaaaaaaaaagctcatattttgtatatatctgcttcagtggagaattatataggttgtgcaaattaacagtcctaactggtatagagcacctagtccagtgacctgctgggtaaactgtggatgatggttgcaaaagactaatttaaaaaataactaccaagaggccctgtctgtacctaacgccctatttttgcaatggctatatggcaagaaagctggtaaactatttgtctttcaggaccttttgaagtagtttgtataacttcttaaaagttgtgattccagataaccagctgtaacacagctgagagacttttaatcagacaaagtaattcctctcactaaactttacccaaaaactaaatctctaatatggcaaaaatggctagacacccattttcacattcccatctgtcaccaattggttaatctttcctgatggtacaggaaagctcagctactgatttttgtgatttagaactgtatgtcagacatccatgtttgtaaaactacacatccctaatgtgtgccatagagtttaacacaagtcctgtgaatttcttcactgttgaaaattattttaaacaaaatagaagctgtagtagccctttctgtgtgcaccttaccaactttctgtaaactcaaaacttaacatatttactaagccacaagaaatttgatttctattcaaggtggccaaattatttgtgtaatagaaaactgaaaatctaatattaaaaatatggaacttctaatatatttttatatttagttatagtttcagatatatatcatattggtattcactaatctgggaagggaagggctactgcagctttacatgcaatttattaaaatgattgtaaaatagcttgtatagtgtaaaataagaatgatttttagatgagattgttttatcatgacatgttatatattttttgtaggggtcaaagaaatgctgatggataacctatatgatttatagtttgtacatgcattcatacaggcagcgatggtctcagaaaccaaacagtttgctctaggggaagagggagatggagactggtcctgtgtgcagtgaaggttgctgaggctctgacccagtgagattacagaggaagttatcctctgcctcccattctgaccacccttctcattccaacagtgagtctgtcagcgcaggtttagtttactcaatctccccttgcactaaagtatgtaaagtatgtaaacaggagacaggaaggtggtgcttacatccttaaaggcaccatctaatagcgggttactttcacatacagccctcccccagcagttgaatgacaacagaagcttcagaagtttggcaatagtttgcatagaggtaccagcaatatgtaaatagtgcagaatctcataggttgccaataatacactaattcctttctatcctacaacaagagtttatttccaaataaaatgaggacatgtttttgttttctttgaatgctttttgaatgttatttgttattttcagtattttggagaaattatttaataaaaaaacaatcatttgctttttg

SEQ ID NO: 2 - DNA, 6322 bp, R. norvegicus
  misc_feature 24, 3663, 3680, 3684, 3685, 3791, 3805, 3806, 3813,3854, 3861, 4162, 4177, 4205, 4206, 4240, 4246, 4247, 4262, 4283,4284, 4293, 4295, 4311, 4354, 4358, 4359, 4360, 4398, 6010,6011, 6013, 6014, 6065, 6069, 6145, 6161: n = A,T,C or G
gacgctgcgggggtgggggacctncggcggcacggagtccccccccgggctcacattaatatttgccaatggactccaaagaatccttagctccccctggtagagacgaagtccctggcagtttgcttggccaagggagggggagcgtaatggacttttataaaagcctgaggggaggagctacagtcaaggtttctgcatcttcgccctcagtggctgctgcttctcaggcagattccaagcagcagaggattctccttgatttctcgaaaggctccacaagcaatgtgcagcagcgacagcagcagcagcagcagcagcagcagcagcagcagcagcagcagcagcagcagcagccaggcttatccaaagccgtttcactgtccatggggctgtatatgggagagacagaaacaaaagtgatggggaatgacttgggctacccacagcagggccaacttggcctttcctctggggaaacagactttcggcttctggaagaaagcattgcaaacctcaataggtcgaccagcgttccagagaaccccaagagttcaacgtctgcaactgggtgtgctaccccgacagagaaggagtttcccaaaactcactcggatgcatcttcagaacagcaaaatcgaaaaagccagaccggcaccaacggaggcagtgtgaaattgtatcccacagaccaaagcacctttgacctcttgaaggatttggagttttccgctgggtccccaagtaaagacacaaacgagagtccctggagatcagatctgttgatagatgaaaacttgctttctcctttggcgggagaagatgatccattccttctcgaagggaacacgaatgaggattgtaagcctcttattttaccggacactaaacctaaaattaaggatactggagatacaatcttatcaagtcccagcagtgtggcactaccccaagtgaaaacagaaaaagatgatttcattgaactttgcacccccggggtaattaagcaagagaaactgggcccagtttattgtcaggcaagcttttctgggacaaatataattggtaataaaatgtctgccatttctgttcatggtgtgagtacctctggaggacagatgtaccactatgacatgaatacagcatccctttctcagcagcaggatcagaagcctgtttttaatgtcattccaccaattcctgttggttctgaaaactggaataggtgccaaggctccggagaggacagcctgacttccttgggggctctgaacttcccaggccggtcagtgttttctaatgggtactcaagccctggaatgagaccagatgtaagctctcctccatccagctcgtcagcagccacgggaccacctcccaagctctgcctggtgtgctccgatgaagcttcaggatgtcattacggggtgctgacatgtggaagctgcaaagtattctttaaaagagcagtggaaggacagcacaattacctttgtgctggaagaaacgattgcatcattgataaaattcgaaggaaaaactgcccagcatgccgctatcggaaatgtcttcaggctggaatgaaccttgaagctcgaaaaacaaagaaaaaaatcaaagggattcagcaagccactgcaggagtctcacaagacacttcggaaaatcctaacaaaacaatagttcctgcagcattaccacagctcacccctaccttggtgtcactgctggaggtgattgaacccgaggtgttgtatgcaggatatgatagctctgttccagattcagcatggagaattatgaccacactcaacatgttaggtgggcgtcaagtgattgcagcagtgaaatgggcaaaggcgatactaggcttgagaaacttacacctcgatgaccaaatgaccctgctacagtactcatggatgtttctcatggcatttgccttgggttggagatcatacagacaatcaagcggaaacctgctctgctttgctcctgatctgattattaatgagcagagaatgtctctaccctgcatgtatgaccaatgtaaacacatgctgtttgtctcctctgaattacaaagattgcaggtatcctatgaagagtatctctgtatgaaaaccttactgcttctctcctcagttcctaaggaaggtctgaagagccaagagttatttgatgagattcgaatgacttatatcaaagagctaggaaaagccatcgtcaaaagggaagggaactccagtcagaactggcaacggttttaccaactgacaaagcttctggactccatgcatgaggtggttgagaatctccttacctactgcttccagacatttttggataagaccatgagtattgaattcccagagatgttagctgaaatcatcactaatcagataccaaaatattcaaatggaaatatcaaaaagcttctgtttcatcaaaaatgactgccttactaagaaaggttgccttaaagaaagttgaatttatagcttttactgtacaaacttatcaatttgtcttgtagatgttttgttgttctttttgtttctgtcttgttttgttttaaacacgcagtacatgtggtttatagagggccaagacttggcgacagaagcagttgagtcaacactctgaagtgatgacacagcacacagtgaagtgtattgttggtgtatcacagaaactaacagttacgtggaggcatggccactgtcagagagggaccgcacctaaaccaccgtgcccaagtccatgtggttcaactttctgactcagaactttacagttggctgggtaaaactttctagactttctgttggtgtatttttcccatgtatagttaggatggtattttgatttatgcatgcaaacctgaaaaaagtttacaagtgtatatcagaaaagggaagttgtgccttttatagctattactgtctggttttaacaatttcctttatattcagtgaactatgcttgctcgtttctcttcaataatttttgtattccagttattgtacagctgtttaagatgggcagctgcttcacagctttcctagacgctaacattaatttccgtgtgaaaatgggtcggtgcttctaccctgttggcaccagctatcagaagaccacagaaattgactcagatctccagtattcttgttaaaaagctcttactctgtatatatctgcttccatggagaattacataggctgagcagattacataggctgagcagattaaccgtcctaactggtgtagagcacctagtccagtgaccttctgggtaaaccgtggatgatggttacagaagactggtgggaaaacagtaactaccaaaaggcccctttccatctaatgcaccatctcttcaatggggagatagcaaccaagcccgtaaatcagctctttcaggaccttctggagtggtttgcataacattttaaaatgtattattccagatagccagctctgataaagccgagagattgtttaatcagaccaagtaacttctctcattaaacttacccccaactaaatcgctaatacagcaagaatggctagacacccattttcacatctcacccgcaccgattggtctagctctcatggtggtcaggagaatcagctactgatttttgttacttagaatnttcaggactcgcatttntccnnctacacatccctacatgtgccatagaatttaacacaagtcctgtgaacttcttcacattgagaattatcattttaaacaaaacagaagcagtagtagccctttctntgtgcaccttaccnnctttctntgactcaaagcttaatatgcttactaagccacaagaaatcngatttcnacttaaaggcgccaaattatttgtgtaatagaaaaactgaaaatctaatattaaaaatatgaaacttctaatatatttttatatttagttatagtttcgatatatatcatatcggtattcactgatcttgggaaagggaaagggctactgcagctttacatgcaatttattaactgactgtaaaatagctgtatagtaataagaatgacttttagtgagattgctttatcatgacatgttatatatttttcgtaggggtcaaagaaatattgatggatatgatagcctatatgatttaatngtatataaaagcatncaaacaggccttaacgcgtcttggaaannaaaatacctttgttctaagctagggaagggagcnggagannggccccgtgtgtatnggaggttccgaggctcggatnnaagagatcnanaggggatctaattccntacctccatctaattacctcaccacccatgatcctgtcagtgnaggnnnggttattaaatcccccgttatactaatataaatagganagaagggtggcgctcacgtctgttccaggcgccgcagtagcagggttattttccatgcagcctcccgacaaggttagcagagggaggctttggcaagtttggcgtggcgtgcatagaggcaccagcaacatgtaaacctaaagagcccataggaagccaagaatacactaatcctccccacccttcaatagtccatttccaagtaagatgaggacatgcttatgttttctttgaatgcttttagaatgttgttattttcagtattttgcagaaattatttaataaaaaagtataatttgaattctctctaaaagggattgttcagtttgtaatggtttaaattggtctcaaagtactttaagataattgtaacccagctggatgtgaaatttatggtgcctaagaaataccacttgaatattatcaagacagtgttaagttttaaaatgagcttctcaaaaatagattattgtacatttatggaatgttatatggttaaacccaaaaaagcacatcacacataaatctgctttcagcttggctttcaaaaatagagctccaaaaacgaaaaaggagaagaaaaagtatatatatgcgttgttattaacagaaggcaacagacattcataaaactactaccgaagctttccttgaagcgtataaagagccatgctcctttagtatgtggggaagaagagagccgtcatagtttcgagtacagagagaagatgcggtactgtctccgtgtgtggcttcataccgttcctaactatttaggtttataataacttcagtgagactcggtgacatgcctgtatgactcatgaccgatcttgaaagatatctttaattactggtaggacaaaagggacactctggttattttaggccttggcttgggatactgtatatccagaagaaaggagacaggaaacttggggaagggaagggaacctaggaagcactgccttctgtaggaaagaacacaccaataagtgagagtacccaaagggacaaggccacacagtgtggggtctaaggatgagtcagggtgagctctggtgggcatggagaagccagcaactccagtgctacagagcagggcagggcagggatgggacaagatggatgcggatcccagtcccagtagtttgctccctcttatttaccatgggatgaaccatggagtattgatctgtcagcactcaaggatcatggagcttgagattccggttggtcaccccaacggtaagctgagattgaatgtgtttcttatgtgccggtttcagtgttagaaggcgaaacagagtgtacagaagacactgcaaaccggtcagatgaaagtcttctcattcccaaactattttcagtcagcctgctctatcaggactggtgaccagctgctaggacagggtcggcgcttctgtctagaatatgcctgaaaggattttattttctgataaatggctgtatgaaaataccctcctcaataacctgcttaactacatagagatttcagtgtgtcaatattctattttgtatattaaacaaaggctatataatggggacaaatctatattatactgtgtatggcattattaagaagcttttnnannattttttatcacagtaatttttaaatgtgtaaaaaattaaaaattagtgantccngtttaaaaataaaagttgtagttttttattcatgctgaataacctgtagtttaaaaatccgtctttctacctacanagtgaaatgtcagacngtaaaattttgtgtggaaatgtttaacttttatttttctttaaatttgctgtcttggtattaccaaaccacacattgtactgaattggcagtaaatgttagtcagccatttacagcaatgccaaatatggataaacatcataataaaatatctgctttttc

SEQ ID NO: 3 - DNA, 2575 bp, M. musculus
ggaagttaatatttgccaatggactccaaagaatccttagctccccctggtagagacgaagtccccagcagtttgcttggccgggggaggggaagcgtgatggacttgtataaaaccctgaggggtggagctacagtcaaggtttctgcgtcttcaccctcagtggctgctgcttctcaggcagattccaagcagcagaggattctccttgatttttcaaaaggctcagcaagcaatgcacagcagcagcagcagcagcagcagccgcagccagatttatccaaagccgtttcactgtccatgggactgtatatgggagagaccgaaacaaaagtgatggggaatgacttgggctacccacagcagggccagcttggcctctcctctggggaaacagactttcggcttctggaagaaagcattgcaaacctcaataggtcgaccagccgtccagagaatcccaagagttcaacacctgcagctgggtgtgctaccccgacagagaaggagtttccccagactcactctgatccatcttcagaacagcaaaatagaaaaagccagcctggcaccaacggtggcagtgtgaaattgtataccacagaccaaagcacctttgacatcttgcaggatttggagttttctgccgggtccccaggtaaagagacaaacgagagtccttggaggtcagacctgttgatagatgaaaacttgctttctcctttggcgggagaagatgatccattccttctggaaggggacgtgaatgaggattgcaagcctcttattttaccggacactaaacctaaaattcaggatactggagatacaatcttatcaagccccagcagtgtggcactgccccaagtgaaaacagagaaagatgatttcattgagctttgcacccctggggtaattaagcaagagaaactgggcccggtttattgccaggcaagcttttctgggacaaatataattgggaataaaatgtctgccatttctgttcatggcgtgagtacctctggaggacagatgtaccactatgacatgaatacagcatccctttctcagcagcaggatcagaagcctgtttttaatgtcattccaccaattcctgttggttctgaaaactggaataggtgccaagggtctggagaggacaacctgacttccttgggggctatgaacttcgcaggccgctcagtgttttctaatggatattcaagccctggaatgagaccagatgtgagttctcctccgtccagctcctccacagcaacgggaccacctcccaaactctgcctggtgtgctccgatgaagcttcggtatgccattatggggtgctgacgtgtggaagctgtaaagtcttctttaaaagagcagtggaaggacagcacaattacctttgtgctggaagaaatgattgcatcattgataaaattcgaagaaaaaactgtccagcatgccgctatcgaaaatgtcttcaagctggaatgaacctggaagctcgaaaaacgaagaaaaaaattaaaggaattcagcaagccactgcaggagtctcacaagacacttctgaaaacgctaacaaaacaatagttcctgccgcgctgccacagcttacccctaccctggtgtcactgctggaggtgatcgagcctgaggtgttatatgcaggatatgacagctctgttccagactcagcatggagaattatgaccacgctcaacatgttaggtgggcgccaagtgattgccgcagtgaaatgggcaaaggcgataccaggattcagaaacttacacctggatgaccaaatgacccttctacagtactcatggatgtttctcatggcatttgccctgggttggagatcatacagacaagcaagtggaaacctgctatgctttgctcctgatctgattattaatgagcagagaatgactctaccctgcatgtatgaccaatgtaaacacatgctgtttatctccactgaattacaaagattgcaggtatcctatgaagagtatctctgtatgaaaaccttactgcttctctcctcagttcctaaggaaggtctgaagagccaagagttatttgatgagattcgaatgacttatatcaaagagctaggaaaagccattgtcaaaagggaaggaaactccagtcagaattggcagcggttttatcaactgacaaaacttttggactccatgcatgatgtggttgaaaatctccttagctactgcttccaaacatttttggataagtccatgagtattgaattcccagagatgttagctgaaatcatcactaatcagataccaaaatactcaaatggaaatatcaaaaagcttctgtttcatcagaaatgactgccttactaagaaaggctgccttaaagaaagttgaatttatagcttttactgtacaaacttatcaacttgtcttgtagatgttttgtcgttctttttgtttgtcttgtttgttttctatacgcactacatgtggtctctagagggccaagacttggcaacagaagcagatgagccatcacttttcagtgacaggaaagcagac

SEQ ID NO: 4 - DNA, 20 bp, Artificial Sequence
  Oligomeric compound
cgtgtgtctgtgctagtccc

SEQ ID NO: 5 - DNA, 20 bp, Artificial Sequence
  Oligomeric compound
ggcaacgtgaacaggtccaa

SEQ ID NO: 6 - DNA, 18 bp, Artificial Sequence
  Oligomeric compound
gcccattgctggacatgc

SEQ ID NO: 7 - DNA, 20 bp, Artificial Sequence
  Oligomeric compound
agcccattgctggacatgca

SEQ ID NO: 8 - DNA, 20 bp, Artificial Sequence
  Oligomeric compound
ttgtcccagtcccaggcctc

SEQ ID NO: 9 - DNA, 20 bp, Artificial Sequence
  Oligomeric compound
ctttccgttggacccctggg

SEQ ID NO: 10 - DNA, 20 bp, Artificial Sequence
  Oligomeric compound
gtgcgcgcgagcccgaaatc

SEQ ID NO: 11 - DNA, 20 bp, Artificial Sequence
  Oligomeric compound
atccaagtgctactgtagta

SEQ ID NO: 12 - DNA, 20 bp, Artificial Sequence
  Oligomeric compound
  misc_feature 1, 2, 3, 4, 5, 6, 7, 8, 9, 10, 11, 12, 13, 14, 15, 16,17, 18, 19, 20: n = A,T,C or G
nnnnnnnnnnnnnnnnnnnn

SEQ ID NO: 13 - DNA, 20 bp, Artificial Sequence
  Oligomeric compound
gccctccatgctggcacagg

SEQ ID NO: 14 - DNA, 20 bp, Artificial Sequence
  Oligomeric compound
agcaaaagatcaatccgtta

SEQ ID NO: 15 - DNA, 20 bp, Artificial Sequence
  Oligomeric compound
tacagaaggctgggccttga

SEQ ID NO: 16 - DNA, 20 bp, Artificial Sequence
  Oligomeric compound
atgcattctgcccccaagga

SEQ ID NO: 17 - DNA, 22 bp, Artificial Sequence
  PCR primer
ttgacattttgcaggatttgga

SEQ ID NO: 18 - DNA, 24 bp, Artificial Sequence
  PCR primer
ccaaggactctcattcgtctcttt

SEQ ID NO: 19 - DNA, 16 bp, Artificial Sequence
  PCR probe
tttcttctgggtcccc

SEQ ID NO: 20 - DNA, 25 bp, Artificial Sequence
  PCR primer
aaacaatagttcctgcagcattacc

SEQ ID NO: 21 - DNA, 23 bp, Artificial Sequence
  PCR primer
catacaacacctcgggttcaatc

SEQ ID NO: 22 - DNA, 23 bp, Artificial Sequence
  PCR probe
acccctaccttggtgtcactgct

SEQ ID NO: 23 - DNA, 23 bp, Artificial Sequence
  PCR primer
gacatcttgcaggatttggagtt

SEQ ID NO: 24 - DNA, 23 bp, Artificial Sequence
  PCR primer
aacaggtctgacctccaaggact

SEQ ID NO: 25 - DNA, 27 bp, Artificial Sequence
  PCR probe
cgggtccccaggtaaagagacaaacga

SEQ ID NO: 26 - DNA, 20 bp, Artificial Sequence
  Oligomeric compound
tctgtctctcccatatacag

SEQ ID NO: 27 - DNA, 20 bp, Artificial Sequence
  Oligomeric compound
tgtttctgtctctcccatat

SEQ ID NO: 28 - DNA, 20 bp, Artificial Sequence
  Oligomeric compound
cttttgtttctgtctctccc

SEQ ID NO: 29 - DNA, 20 bp, Artificial Sequence
  Oligomeric compound
atcacttttgtttctgtctc

SEQ ID NO: 30 - DNA, 20 bp, Artificial Sequence
  Oligomeric compound
gtttgcaatgctttcttcca

SEQ ID NO: 31 - DNA, 20 bp, Artificial Sequence
  Oligomeric compound
tgaggtttgcaatgctttct

SEQ ID NO: 32 - DNA, 20 bp, Artificial Sequence
  Oligomeric compound
ctattgaggtttgcaatgct

SEQ ID NO: 33 - DNA, 20 bp, Artificial Sequence
  Oligomeric compound
cgacctattgaggtttgcaa

SEQ ID NO: 34 - DNA, 20 bp, Artificial Sequence
  Oligomeric compound
ctggtcgacctattgaggtt

SEQ ID NO: 35 - DNA, 20 bp, Artificial Sequence
  Oligomeric compound
ctgtggtatacaatttcaca

SEQ ID NO: 36 - DNA, 20 bp, Artificial Sequence
  Oligomeric compound
ctttggtctgtggtatacaa

SEQ ID NO: 37 - DNA, 20 bp, Artificial Sequence
  Oligomeric compound
gtcaaaggtgctttggtctg

SEQ ID NO: 38 - DNA, 20 bp, Artificial Sequence
  Oligomeric compound
ggtttagtgtccggtaaaat

SEQ ID NO: 39 - DNA, 20 bp, Artificial Sequence
  Oligomeric compound
ctttttctgttttcacttgg

SEQ ID NO: 40 - DNA, 20 bp, Artificial Sequence
  Oligomeric compound
ttctcttgcttaattacccc

SEQ ID NO: 41 - DNA, 20 bp, Artificial Sequence
  Oligomeric compound
cagtttctcttgcttaatta

SEQ ID NO: 42 - DNA, 20 bp, Artificial Sequence
  Oligomeric compound
gcccagtttctcttgcttaa

SEQ ID NO: 43 - DNA, 20 bp, Artificial Sequence
  Oligomeric compound
tttattaccaattatatttg

SEQ ID NO: 44 - DNA, 20 bp, Artificial Sequence
  Oligomeric compound
acattttattaccaattata

SEQ ID NO: 45 - DNA, 20 bp, Artificial Sequence
  Oligomeric compound
gcagacattttattaccaat

SEQ ID NO: 46 - DNA, 20 bp, Artificial Sequence
  Oligomeric compound
aatggcagacattttattac

SEQ ID NO: 47 - DNA, 20 bp, Artificial Sequence
  Oligomeric compound
cagaaatggcagacatttta

SEQ ID NO: 48 - DNA, 20 bp, Artificial Sequence
  Oligomeric compound
tgaacagaaatggcagacat

SEQ ID NO: 49 - DNA, 20 bp, Artificial Sequence
  Oligomeric compound
ccatgaacagaaatggcaga

SEQ ID NO: 50 - DNA, 20 bp, Artificial Sequence
  Oligomeric compound
cacaccatgaacagaaatgg

SEQ ID NO: 51 - DNA, 20 bp, Artificial Sequence
  Oligomeric compound
tactcacaccatgaacagaa

SEQ ID NO: 52 - DNA, 20 bp, Artificial Sequence
  Oligomeric compound
gaggtactcacaccatgaac

SEQ ID NO: 53 - DNA, 20 bp, Artificial Sequence
  Oligomeric compound
tccagaggtactcacaccat

SEQ ID NO: 54 - DNA, 20 bp, Artificial Sequence
  Oligomeric compound
gtcctccagaggtactcaca

SEQ ID NO: 55 - DNA, 20 bp, Artificial Sequence
  Oligomeric compound
atctgtcctccagaggtact

SEQ ID NO: 56 - DNA, 20 bp, Artificial Sequence
  Oligomeric compound
gtacatctgtcctccagagg

SEQ ID NO: 57 - DNA, 20 bp, Artificial Sequence
  Oligomeric compound
agtggtacatctgtcctcca

SEQ ID NO: 58 - DNA, 20 bp, Artificial Sequence
  Oligomeric compound
tcatagtggtacatctgtcc

SEQ ID NO: 59 - DNA, 20 bp, Artificial Sequence
  Oligomeric compound
catgtcatagtggtacatct

SEQ ID NO: 60 - DNA, 20 bp, Artificial Sequence
  Oligomeric compound
tattcatgtcatagtggtac

SEQ ID NO: 61 - DNA, 20 bp, Artificial Sequence
  Oligomeric compound
gctgtattcatgtcatagtg

SEQ ID NO: 62 - DNA, 20 bp, Artificial Sequence
  Oligomeric compound
ggatgctgtattcatgtcat

SEQ ID NO: 63 - DNA, 20 bp, Artificial Sequence
  Oligomeric compound
aaagggatgctgtattcatg

SEQ ID NO: 64 - DNA, 20 bp, Artificial Sequence
  Oligomeric compound
tgagaaagggatgctgtatt

SEQ ID NO: 65 - DNA, 20 bp, Artificial Sequence
  Oligomeric compound
tggtggaatgacattaaaaa

SEQ ID NO: 66 - DNA, 20 bp, Artificial Sequence
  Oligomeric compound
gaattggtggaatgacatta

SEQ ID NO: 67 - DNA, 20 bp, Artificial Sequence
  Oligomeric compound
gagcttacatctggtctcat

SEQ ID NO: 68 - DNA, 20 bp, Artificial Sequence
  Oligomeric compound
aggagagcttacatctggtc

SEQ ID NO: 69 - DNA, 20 bp, Artificial Sequence
  Oligomeric compound
atggaggagagcttacatct

SEQ ID NO: 70 - DNA, 20 bp, Artificial Sequence
  Oligomeric compound
ctggatggaggagagcttac

SEQ ID NO: 71 - DNA, 20 bp, Artificial Sequence
  Oligomeric compound
gagctggatggaggagagct

SEQ ID NO: 72 - DNA, 20 bp, Artificial Sequence
  Oligomeric compound
tgtccttccactgctctttt

SEQ ID NO: 73 - DNA, 20 bp, Artificial Sequence
  Oligomeric compound
gtgctgtccttccactgctc

SEQ ID NO: 74 - DNA, 20 bp, Artificial Sequence
  Oligomeric compound
aattgtgctgtccttccact

SEQ ID NO: 75 - DNA, 20 bp, Artificial Sequence
  Oligomeric compound
aggtaattgtgctgtccttc

SEQ ID NO: 76 - DNA, 20 bp, Artificial Sequence
  Oligomeric compound
cggcatgctgggcagttttt

SEQ ID NO: 77 - DNA, 20 bp, Artificial Sequence
  Oligomeric compound
atagcggcatgctgggcagt

SEQ ID NO: 78 - DNA, 20 bp, Artificial Sequence
  Oligomeric compound
cgatagcggcatgctgggca

SEQ ID NO: 79 - DNA, 20 bp, Artificial Sequence
  Oligomeric compound
attccagcctgaagacattt

SEQ ID NO: 80 - DNA, 20 bp, Artificial Sequence
  Oligomeric compound
gttcattccagcctgaagac

SEQ ID NO: 81 - DNA, 20 bp, Artificial Sequence
  Oligomeric compound
ttctttgtttttcgagcttc

SEQ ID NO: 82 - DNA, 20 bp, Artificial Sequence
  Oligomeric compound
ttttttctttgtttttcgag

SEQ ID NO: 83 - DNA, 20 bp, Artificial Sequence
  Oligomeric compound
caggaactattgttttgtta

SEQ ID NO: 84 - DNA, 20 bp, Artificial Sequence
  Oligomeric compound
tgcaggaactattgttttgt

SEQ ID NO: 85 - DNA, 20 bp, Artificial Sequence
  Oligomeric compound
gagctatcatatcctgcata

SEQ ID NO: 86 - DNA, 20 bp, Artificial Sequence
  Oligomeric compound
aacagagctatcatatcctg

SEQ ID NO: 87 - DNA, 20 bp, Artificial Sequence
  Oligomeric compound
ctggaacagagctatcatat

SEQ ID NO: 88 - DNA, 20 bp, Artificial Sequence
  Oligomeric compound
ttcactgctgcaatcacttg

SEQ ID NO: 89 - DNA, 20 bp, Artificial Sequence
  Oligomeric compound
ccatttcactgctgcaatca

SEQ ID NO: 90 - DNA, 20 bp, Artificial Sequence
  Oligomeric compound
ttgcccatttcactgctgca

SEQ ID NO: 91 - DNA, 20 bp, Artificial Sequence
  Oligomeric compound
ataatcagatcaggagcaaa

SEQ ID NO: 92 - DNA, 20 bp, Artificial Sequence
  Oligomeric compound
attaataatcagatcaggag

SEQ ID NO: 93 - DNA, 20 bp, Artificial Sequence
  Oligomeric compound
gctcattaataatcagatca

SEQ ID NO: 94 - DNA, 20 bp, Artificial Sequence
  Oligomeric compound
ctctgctcattaataatcag

SEQ ID NO: 95 - DNA, 20 bp, Artificial Sequence
  Oligomeric compound
cattctctgctcattaataa

SEQ ID NO: 96 - DNA, 20 bp, Artificial Sequence
  Oligomeric compound
agcatgtgtttacattggtc

SEQ ID NO: 97 - DNA, 20 bp, Artificial Sequence
  Oligomeric compound
aaggttttcatacagagata

SEQ ID NO: 98 - DNA, 20 bp, Artificial Sequence
  Oligomeric compound
cagtaaggttttcatacaga

SEQ ID NO: 99 - DNA, 20 bp, Artificial Sequence
  Oligomeric compound
gaagcagtaaggttttcata

SEQ ID NO: 100 - DNA, 20 bp, Artificial Sequence
  Oligomeric compound
gagagaagcagtaaggtttt

SEQ ID NO: 101 - DNA, 20 bp, Artificial Sequence
  Oligomeric compound
gcttttcctagctctttgat

SEQ ID NO: 102 - DNA, 20 bp, Artificial Sequence
  Oligomeric compound
atggcttttcctagctcttt

SEQ ID NO: 103 - DNA, 20 bp, Artificial Sequence
  Oligomeric compound
atggtcttatccaaaaatgt

SEQ ID NO: 104 - DNA, 20 bp, Artificial Sequence
  Oligomeric compound
actcatggtcttatccaaaa

SEQ ID NO: 105 - DNA, 20 bp, Artificial Sequence
  Oligomeric compound
caatactcatggtcttatcc

SEQ ID NO: 106 - DNA, 20 bp, Artificial Sequence
  Oligomeric compound
aattcaatactcatggtctt

SEQ ID NO: 107 - DNA, 20 bp, Artificial Sequence
  Oligomeric compound
atgatttcagctaacatctc

SEQ ID NO: 108 - DNA, 20 bp, Artificial Sequence
  Oligomeric compound
gtgatgatttcagctaacat

SEQ ID NO: 109 - DNA, 20 bp, Artificial Sequence
  Oligomeric compound
gaatattttggtatctgatt

SEQ ID NO: 110 - DNA, 20 bp, Artificial Sequence
  Oligomeric compound
atttgaatattttggtatct

SEQ ID NO: 111 - DNA, 20 bp, Artificial Sequence
  Oligomeric compound
ttccatttgaatattttggt

SEQ ID NO: 112 - DNA, 20 bp, Artificial Sequence
  Oligomeric compound
atatttccatttgaatattt

SEQ ID NO: 113 - DNA, 20 bp, Artificial Sequence
  Oligomeric compound
tttttgatatttccatttga

SEQ ID NO: 114 - DNA, 20 bp, Artificial Sequence
  Oligomeric compound
ccttccctgaaggttcctcc